(19) United States
(12) Patent Application Publication
(10) Pub. No.: US 2023/0218716 A1
(43) Pub. Date: Jul. 13, 2023

(54) COMPOSITIONS AND METHODS FOR TREATING ATRIAL FIBRILLATION

(71) Applicant: Ohio State Innovation Foudation, Columbus, OH (US)

(72) Inventors: Rengasayee VEERARAGHAVAN, Bexley, OH (US); Louisa MEZACHE, Columbus, OH (US)

(21) Appl. No.: 17/997,666

(22) PCT Filed: May 6, 2021
(86) PCT No.: PCT/US2021/031032

§ 371 Date: Nov. 1, 2022

Related U.S. Application Data
(60) Provisional application No. 63/020,880, filed on May 6, 2020.

(51) Int. Cl. A61K 38/17; G01N 33/68; A61K 31/4709

(52) U.S. Cl. CPC A61K 38/1709; G01N 33/68; A61K 31/4709

(57) ABSTRACT

Disclosed herein is a method for treating atrial fibrillation (AF) or reentrant ventricular arrhythmias in a subject that involves administering to the subject a therapeutically effective amount of a gap junction or pannexin channel inhibitor in an amount effective to preserve barrier function. In some embodiments, the subject has paroxysmal AF.

### CROSS-REFERENCE TO RELATED APPLICATIONS

This application claims benefit of U.S. Provisional Application No. 63/020,880, filed May 6, 2020, which is hereby incorporated herein by reference in its entirety.

### SEQUENCE LISTING

This application contains a sequence listing filed in electronic form as an ASCII.txt file entitled “321501_2460_PCT_Patent_Application_ST25” created on Mar. 1, 2021. The content of the sequence listing is incorporated herein in its entirety.

### BACKGROUND

Atrial fibrillation (AF) is the most common sustained cardiac arrhythmia in clinical practice and is known to be associated with significant morbidity and mortality. Previous studies suggested a link between inflammation and AF, finding increased inflammatory markers in AF patients. However, it has not been finally clarified how inflammation, occurring systemically or as a local phenomenon in the heart, contributes to the development and progression of AF. More importantly, the development of preventative therapies for AF has been disappointing. Likewise, inflammation has been linked to reentrant ventricular arrhythmias in multiple pathologies, although the underlying mechanistic link has not been fully clarified.

### SUMMARY

Disclosed herein is a method for treating inflammation-induced vascular leak and consequent cardiac arrhythmia in a subject that involves administering to the subject a therapeutically effective amount of a gap junction hemichannel or pannexin channel inhibitor to preserve barrier function. In some embodiments, inhibiting hemichannels, which connect the inside of the cell with the extracellular space, can be anti-arrhythmic. In contrast, a drug that inhibits inter-cellular gap junctions may prove proarrhythmic.

Inflammation-induced vascular leak and consequent arrhythmias are a common feature of multiple pathologies. Early stage AF patients have elevated levels of inflammatory cytokines, such as interleukin-6 (IL-6), vascular endothelial growth factor (VEGF) and tumor necrosis factor α (TNFα). IL-6 often functions as an upstream regulator of vascular leak-inducing cytokines such as VEGF and TNFα, and in cardiac myocytes, it induces signaling via the mitogen-activated protein kinase (MAPK) pathway. In turn, MAPK signaling, specifically mediated by p38α MAPK, induces production of IL-6, VEGF and TNFα by cardiac myocytes. Thus, the IL-6-MAPK signaling axis may be a positive feedback loop that links over-recruitment of inflammation with excessive vascular leak (via VEGF, TNFα etc) and cardiac arrhythmias. Vascular leak induces such arrhythmias via nanoscale damage to intercalated disks, specialized structures that provide electrical and mechanical coupling between cardiac myocytes. In addition to atrial fibrillation, this mechanism is also common to ventricular arrhythmias in myocardial infarction, diabetes, and in heart failure. The proposed arrhythmia mechanism and treatment strategy are therefore applicable to any pathology associated with inflammation, vascular leak and cardiac arrhythmias. In some embodiments, the cardiac arrhythmia is an atrial fibrillation (AF). In some embodiments, the subject has paroxysmal AF. Paroxysmal AF are episodes of AF that occur occasionally and usually stop spontaneously. Episodes can last a few seconds, hours or a few days before stopping and returning to normal sinus rhythm, which is the heart's normal rhythm. In some embodiments, the subject has reentrant ventricular arrhythmias, which can be immediately life-threatening, if left untreated.

Also disclosed herein is a biomarker of arrhythmias caused by inflammation-induced vascular leak. The ectodomain of the sodium channel auxiliary subunit β1 is a serum biomarker for arrhythmias resulting from inflammation-induced intercalated disk damage. The sodium channel auxiliary subunit β1 provides adhesion within gap junction-adjacent perinexal sites within the intercalated disk. Vascular leak-induced cardiac edema led to de-adhesion at these sites and ventricular as well as atrial arrhythmias. Super-resolution microscopy revealed loss of β1 from these locations during such de-adhesion. Notably, Naᵥβ subunits (β1, β2, and β4) undergo ectodomain shedding and regulated intramembrane proteolysis following cleavage by the enzymes β-secretase (BACE1) and γ-secretase (presenilin). The sequence adjacent to the transmembrane domain on each Naᵥβ subunit contains a putative BACE1 cleavage site(s), and the N-terminal part of VGSCβ is shed and released similar to that of amyloid plaque protein. While much of the research into Naᵥβ cleavage was conducted in neurons, β1 is known to be cleaved via these mechanisms in the heart. Therefore, the β1 ectodomain can be exploited as a serum biomarker for pro-arrhythmic intercalated disk damage. Arrhythmias under these conditions can be prevented using the disclosed methods.

In some embodiments, the β1 ectodomain comprises amino acids 44-60 of the full-length protein. Therefore, in some embodiments, β1 ectodomain comprises the amino acid sequence KRRSETTAETFTEWTFR (SEQ ID NO:1). In some embodiments, this β1 ectodomain can be detected by an antibody that selectively binds SEQ ID NO:1. Antibodies that can be used in the disclosed compositions and methods include whole immunoglobulin (i.e., an intact antibody) of any class, fragments thereof, and synthetic proteins containing at least the antigen binding variable domain of an antibody.

In some embodiments, the gap junction hemichannel inhibitor is a connexin43 hemichannel inhibitor. For example, in some cases, the connexin43 hemichannel inhibitor is a polypeptide comprising from 4 to 30 contiguous amino acids of the carboxy-terminus of the alpha Connexin (e.g. αCT11). In some embodiments, the gap junction hemichannel inhibitor is mefloquine. In some embodiments, the connexin43 hemichannel inhibitor is selected from the group consisting of JM2, Gap19 (intracellular loop), Gap26 (extracellular loop 1), Gap27 (extracellular loop 2), a trivalent cation (e.g. La³⁺, Gd³⁺), Niflumic acid, Heptanol, Meclofenamic acid, Digoxin, PDBu, Lindane, Glycyrrhizin agents, Carbenoxolone, 18α-GA, 18β-GA, Flufenamic acid, Octanol, Halothane, Linoleic acid, Oleic acid, Arachidonic acid, Mefloquine, 2-APB, Polyamines, and Tonabersat.

In some embodiments, the pannexin-1 channel inhibitor is a Panx1-IL2 peptide. In some embodiments, the pannexin-1 channel inhibitor is spironolactone. In some embodiments, the pannexin-1 channel inhibitor is selected from the group consisting of probenecid, carbenoxolone, glycyrrhizin agents, arachidonic acid, and brilliant blue FCF.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. Acute effects of VEGF on atrial conduction. A) Representative volume-conducted ECGs. B) Summary plots of P wave duration (n=5/group; *p<0.05 vs. control). C) Representative isochrone maps of left atrial activation. D) Summary plots of CV (n=5/group; *p<0.05 vs. control).

FIG. 2. Acute impact of VEGF on atrial arrhythmia susceptibility. A) Representative volume-conducted ECGs show response to burst pacing. B) Incidence of atrial arrhythmias following burst pacing (n=5/group, *p<0.05 vs. control). C) Representative in vivo surface ECG illustrates atrial arrhythmia observed in a VEGF-treated mouse. D) Total atrial arrhythmia burden quantified as seconds of arrhythmia per hour of observation (n=10/group, *p<0.05 vs. control).

FIG. 3. VEGF effects on expression of ID proteins. A) Western immunoblots and B) summary quantification of ID protein expression from VEGF-treated and vehicle control hearts (n=3/group, *p<0.05 vs. control).

FIG. 4. VEGF effects on ID ultrastructure. A) Representative TEM images of IDs. B) Summary plots of intermembrane distance at GJ-adjacent perinexal sites (solid bars) and MJ-adjacent (striped bars) ID sites (>100 measurements/group/location from n=3 hearts/group, *p<0.05 vs. control).

FIG. 5. sDCI imaging of IDs. Representative 3D sDCI images of en face IDs from murine atria immunolabeled for A, B) Naᵥ1.5, Cx40, Cx43, and N-cad, and C, D) Naᵥ1.5, β1, Cx43, and N-cad.

FIG. 6. STED imaging of atrial IDs. Representative 3D STED images of en face IDs from VEGF-treated and control murine atria immunolabeled for A) Naᵥ1.5 and B) β1 along with Cx43 and N-cad.

FIG. 7. OBS3D analysis of STED images. A) Bivariate histograms of Naᵥ1.5 cluster mass (normalized intensity summed over the cluster) as a function of distance from Cx43 clusters. These provide representative examples of intermediate steps in image analysis involved in assessing enrichment ratios, calculated as the ratio of Naᵥ1.5/β1 signal intensity within 100 nm of Cx43 (GJ) and N-cad (MJ) clusters to Naᵥ1.5/β1 density at other ID sites. B) Summary plots of enrichment ratio (n=3 hearts/group, 3 images/heart; *p<0.05 vs. control).

FIG. 8. STORM imaging of atrial IDs—Control hearts. Representative 3D STORM images of en face IDs from control murine atria immunolabeled for Naᵥ1.5 and β1 along with Cx43 and N-cad. STORM data are rendered as point clouds with each localized molecule represented as a 50 nm sphere. Although 20 nm resolution was achieved, the 50 nm size was chosen for rendering to guarantee visibility in print.

FIG. 9. STORM imaging of atrial IDs—VEGF-treated hearts. Representative 3D STORM images of en face IDs from VEGF-treated murine atria immunolabeled for Naᵥ1.5 and β1 along with Cx43 and N-cad.

FIG. 10. STORM-RLA analysis of Naᵥ1.5, β1 localization. Representative 3D STORM images of a Cx43 cluster and associated Naᵥ1.5 clusters from A) control and B) VEGF-treated murine atria. C, D) Bivariate histograms of Naᵥ1.5 cluster density as a function of distance from Cx43 clusters. Dashed circles highlight the decrease in Naᵥ1.5 clusters located near Cx43. E) Summary plots of STORM-RLA results. Left: % of ID-localized Naᵥ1.5 and β1 located within 100 nm of Cx43 (GJ) and N-cad (MJ) clusters. Right: Enrichment ratio, calculated as the ratio of Naᵥ1.5/β1 density within 100 nm of Cx43 (GJ) and N-cad (MJ) clusters to Naᵥ1.5/β1 density at other ID sites (n=3 hearts/group, 10 images/heart; *p<0.05 vs. control).

FIG. 11. Proposed mechanism for the genesis and progression of AF. Elevated VEGF levels in AF patients increase vascular leak, in turn promoting cardiac edema. The resulting disruption of Naᵥ1.5-rich ID nanodomains slows atrial conduction, thereby providing a substrate for further atrial arrhythmias.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “prevent” refers to a treatment that forestalls or slows the onset of a disease or condition or reduced the severity of the disease or condition. Thus, if a treatment can treat a disease in a subject having symptoms of the disease, it can also prevent that disease in a subject who has yet to suffer some or all of the symptoms.

The term “agent” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. For example, an agent can be an oligomer of nucleic acids, amino acids, or carbohydrates including, but not limited to proteins, peptides, oligonucleotides, ribozymes, DNAzymes, glycoproteins, RNAi agents (e.g., siRNAs), lipoproteins, aptamers, and modifications and combinations thereof. In some embodiments, an active agent is a nucleic acid, e.g., miRNA or a derivative or variant thereof.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “atrial fibrillation” or “AF” refers to a condition where the heart's two upper chambers (the right and left atria) quiver instead of beating and contracting rhythmically. Electrocardiographically, AF is characterized by a highly disorganized atrial electrical activity that often results in fast beating of the heart's two lower chambers (the right and left ventricles). Symptoms experienced by patients with AF include palpitation, fatigue, and dyspnea (shortness of breath). There are three types of AF based on the presentation and duration of the arrhythmia: a) Paroxysmal AF: recurrent AF (>2 episodes) that starts and terminates spontaneously within 7 days (paroxysmal AF starts and stops spontaneously); b) Persistent AF: sustained AF that lasts longer than 7 days or requires termination by pharmacologic or electrical cardioversion (electrical shock); and c) Permanent AF: long standing AF (for >1 year duration) in which normal sinus rhythm cannot be maintained even after treatment, or when the patient and physician have decided to allow AF to continue without further efforts to restore sinus rhythm.

The term “atrial flutter” refers to an abnormal heart rhythm that occurs in the atria of the heart. When it first occurs, it is usually associated with a fast heart rate or tachycardia (230-380 beats per minute (bpm)), and falls into the category of supra-ventricular tachycardias. While this rhythm occurs most often in individuals with cardiovascular disease (e.g. hypertension, coronary artery disease, and cardiomyopathy), it may occur spontaneously in people with otherwise normal hearts. It is typically not a stable rhythm, and frequently degenerates into atrial fibrillation (AF).

The term “reentrant ventricular arrhythmia” refers to a type of paroxysmal tachycardia occurring in the ventricle where the cause of the arrhythmia is due to the electric signal not completing the normal circuit, but rather an alternative circuit looping back upon itself.

### Gap Junction Inhibitor

In some embodiments, the gap junction hemichannel inhibitor is a connexin43 hemichannel inhibitor. For example, in some cases, the connexin43 hemichannel inhibitor is a polypeptide comprising from 4 to 30 contiguous amino acids of the carboxy-terminus of the alpha Connexin.

For example, in some embodiments, the a connexin43 hemichannel inhibitor is an alpha connexin c-terminal (ACT) peptide disclosed in U.S. Pat. No. 10,398,757, which is incorporated by reference in its entirety for the description of these peptides, methods of making these peptides, and pharmaceutical compositions containing these peptides.

The herein provided polypeptide can be any polypeptide comprising the carboxy-terminal most amino acids of an alpha Connexin, wherein the polypeptide does not comprise the full-length alpha Connexin protein. Thus, in one aspect, the provided polypeptide does not comprise the cytoplasmic N-terminal domain of the alpha Connexin. In another aspect, the provided polypeptide does not comprise the two extracellular domains of the alpha Connexin. In another aspect, the provided polypeptide does not comprise the four transmembrane domains of the alpha Connexin. In another aspect, the provided polypeptide does not comprise the cytoplasmic loop domain of the alpha Connexin. In another aspect, the provided polypeptide does not comprise that part of the sequence of the cytoplasmic carboxyl terminal domain of the alpha Connexin proximal to the fourth transmembrane domain. There is a conserved proline or glycine residue in alpha Connexins consistently positioned some 17 to 30 amino acids from the carboxyl terminal-most amino acid. For example, for human Cx43 a proline residue at amino acid 363 is positioned 19 amino acids back from the carboxyl terminal most isoleucine. In another example, for chick Cx43 a proline residue at amino acid 362 is positioned 18 amino acids back from the carboxyl terminal-most isoleucine. In another example, for human Cx45 a glycine residue at amino acid 377 is positioned 19 amino acids back from the carboxyl terminal most isoleucine. In another example for rat Cx33, a proline residue at amino acid 258 is positioned 28 amino acids back from the carboxyl terminal most methionine. Thus, in another aspect, the provided polypeptide does not comprise amino acids proximal to said conserved proline or glycine residue of the alpha Connexin. Thus, the provided polypeptide can comprise the c-terminal-most 4 to 30 amino acids of the alpha Connexin, including the c-terminal most 4, 5, 6, 7, 8, 9, 10, 11, 12, 13, 14, 15, 16, 17, 18, 19, 20, 21, 22, 23, 24, 25, 26, 27, 28, 29, 30 amino acids of the alpha Connexin.

The carboxy-terminal most amino acids of an alpha Connexin in the provided peptides can be flanked by non-alpha Connexin or non-ACT peptide Connexin amino acids. Examples of the flanking non-alpha Connexin and non-ACT Connexin amino acids are provided herein. An example of non-ACT Connexin amino acids are the carboxy-terminal 20 to 120 amino acids of human Cx43 (KTDPYSHSGTMSPSKDCGSPKYAYYNGCSSPTAPLSPMSPPGYKLVTGDRNNSSCRNYN KQASEQNWANYSAEQNRMGQAGSTISNSHAQPFDFADEHQNTKKLASGHELQPLTIVDQR P, SEQ ID NO:16).

An example of a non-alpha Connexin is the 239 amino acid sequence of enhanced green fluorescent protein. In another aspect, given that ACT1 is shown to be functional when fused to the carboxy terminus of the 239 amino acid sequence of GFP, ACT peptides are expected to retain function when flanked with non-Connexin polypeptides of up to at least 239 amino acids. Indeed, as long as the ACT sequence is maintained as the free carboxy terminus of a given polypeptide, and the ACT peptide is able to access its targets. Thus, polypeptides exceeding 239 amino acids in addition to the ACT peptide can function in reducing inflammation, promoting healing, increasing tensile strength, reducing scarring and promoting tissue regeneration following injury.

Connexins are the sub-unit protein of the gap junction channel which is responsible for intercellular communication. Based on patterns of conservation of nucleotide sequence, the genes encoding Connexin proteins are divided into two families termed the alpha and beta Connexin genes. The carboxy-terminal-most amino acid sequences of alpha Connexins are characterized by multiple distinctive and conserved features. This conservation of organization is consistent with the ability of ACT peptides to form distinctive 3D structures, interact with multiple partnering proteins, mediate interactions with lipids and membranes, interact with nucleic acids including DNA, transit and/or block membrane channels and provide consensus motifs for proteolytic cleavage, protein cross-linking, ADP-ribosylation, glycosylation and phosphorylation. Thus, the provided polypeptide interacts with a domain of a protein that normally mediates the binding of said protein to the carboxy-terminus of an alpha Connexin. For example, nephroblastoma overexpressed protein (NOV) interacts with a Cx43 c-terminal domain. It is considered that this and other proteins interact with the carboxy-terminus of alpha Connexins and further interact with other proteins forming a macromolecular complex. Thus, the provided polypeptide can inhibit the operation of a molecular machine, such as, for example, one involved in regulating the aggregation of Cx43 gap junction channels.

As used herein, “inhibit,” “inhibiting,” and “inhibition” mean to decrease an activity, response, condition, disease, or other biological parameter. This can include, but is not limited to, the complete loss of activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The ACT sequence of the provided polypeptide can be from any alpha Connexin. Thus, the alpha Connexin component of the provided polypeptide can be from a human, murine, bovine, monotrene, marsupial, primate, rodent, cetacean, mammalian, avian, reptilian, amphibian, piscine, chordate, protochordate or other alpha Connexin.

Thus, the provided polypeptide can comprise an ACT of a Connexin selected from the group consisting of mouse Connexin 47, human Connexin 47, Human Connexin 46.6, Cow Connexin 46.6, Mouse Connexin 30.2, Rat Connexin 30.2, Human Connexin 31.9, Dog Connexin 31.9, Sheep Connexin 44, Cow Connexin 44, Rat Connexin 33, Mouse Connexin 33, Human Connexin 36, mouse Connexin 36, rat Connexin 36, dog Connexin 36, chick Connexin 36, zebrafish Connexin 36, morone Connexin 35, morone Connexin 35, Cynops Connexin 35, Tetraodon Connexin 36, human Connexin 37, chimp Connexin 37, dog Connexin 37, Cricetulus Connexin 37, Mouse Connexin 37, Mesocricetus Connexin 37, Rat Connexin 37, mouse Connexin 39, rat Connexin 39, human Connexin 40.1, Xenopus Connexin 38, Zebrafish Connexin 39.9, Human Connexin 40, Chimp Connexin 40, dog Connexin 40, cow Connexin 40, mouse Connexin 40, rat Connexin 40, Cricetulus Connexin 40, Chick Connexin 40, human Connexin43, Cercopithecus Connexin43, Oryctolagus Connexin43, Spermophilus Connexin43, Cricetulus Connexin43, Phodopus Connexin43, Rat Connexin43, Sus Connexin43, Mesocricetus Connexin43, Mouse Connexin43, Cavia Connexin43, Cow Connexin43, Erinaceus Connexin43, Chick Connexin43, Xenopus Connexin43, Oryctolagus Connexin43, Cyprinus Connexin43, Zebrafish Connexin43, Danio aequipinnatus Connexin43, Zebrafish Connexin43.4, Zebrafish Connexin 44.2, Zebrafish Connexin 44.1, human Connexin 45, chimp Connexin 45, dog Connexin 45, mouse Connexin 45, cow Connexin 45, rat Connexin 45, chick Connexin 45, Tetraodon Connexin 45, chick Connexin 45, human Connexin 46, chimp Connexin 46, mouse Connexin 46, dog Connexin 46, rat Connexin 46, Mesocricetus Connexin 46, Cricetulus Connexin 46, Chick Connexin 56, Zebrafish Connexin 39.9, cow Connexin 49, human Connexin 50, chimp Connexin 50, rat Connexin 50, mouse Connexin 50, dog Connexin 50, sheep Connexin 49, Mesocricetus Connexin 50, Cricetulus Connexin 50, Chick Connexin 50, human Connexin 59, or other alpha Connexin.

The 20-30 carboxy-terminal-most amino acid sequence of alpha Connexins are characterized by a distinctive and conserved organization. This distinctive and conserved organization would include a type II PDZ binding motif (ϕ-x-ϕ; wherein x=any amino acid and ϕ=a Hydrophobic amino acid) and proximal to this motif, Proline (P) and/or Glycine (G) hinge residues; a high frequency phospho-Serine (S) and/or phospho-Threonine (T) residues; and a high frequency of positively charged Arginine (R), Lysine (K) and negatively charged Aspartic acid (D) or Glutamic acid (E) amino acids. For many alpha Connexins, the P and G residues occur in clustered motifs proximal to the carboxy-terminal type II PDZ binding motif. The S and T phosphor-amino acids of most alpha Connexins also are typically organized in clustered, repeat-like motifs.

Thus, in one aspect, the provided polypeptide comprises one, two, three or all of the amino acid motifs selected from the group consisting of 1) a type II PDZ binding motif, 2) Proline (P) and/or Glycine (G) hinge residues; 3) clusters of phospho-Serine (S) and/or phospho-Threonine (T) residues; and 4) a high frequency of positively charged Arginine (R) and Lysine (K) and negatively charged Aspartic acid (D) and/or Glutamic acid (E) amino acids). In another aspect, the provided polypeptide comprises a type II PDZ binding motif at the carboxy-terminus, Proline (P) and/or Glycine (G) hinge residues proximal to the PDZ binding motif, and positively charged residues (K, R, D, E) proximal to the hinge residues.

PDZ domains were originally identified as conserved sequence elements within the postsynaptic density protein PSD95/SAP90, the Drosophila tumor suppressor dig-A, and the tight junction protein ZO-1. Although originally referred to as GLGF or DHR motifs, they are now known by an acronym representing these first three PDZ-containing proteins (PSD95/DLG/ZO-1). These 80-90 amino acid sequences have now been identified in well over 75 proteins and are characteristically expressed in multiple copies within a single protein. Thus, in one aspect, the provided polypeptide can inhibit the binding of an alpha Connexin to a protein comprising a PDZ domain. The PDZ domain is a specific type of protein-interaction module that has a structurally well-defined interaction ‘pocket’ that can be filled by a PDZ-binding motif, referred to herein as a “PDZ motif”. PDZ motifs are consensus sequences that are normally, but not always, located at the extreme intracellular carboxyl terminus. Four types of PDZ motifs have been classified: type I (S/T-x-ϕ), type II (ϕ-x-ϕ), type III (ψ-xϕ) and type IV (D-x-V), where x is any amino acid, ϕ is a hydrophobic residue (V, I, L, A, G, W, C, M, F) and ψ is a basic, hydrophilic residue (H, R, K). (Songyang, Z., et al. 1997. Science 275, 73-77). Thus, in one aspect, the provided polypeptide comprises a type II PDZ binding motif.

In some embodiments, the provided polypeptide comprises a type II PDZ binding motif (ϕ-xϕ; wherein x=any amino acid and ϕ=a Hydrophobic amino acid). In another aspect, greater than 50%, 60%, 70%, 80%, 90% of the amino acids of the provided ACT polypeptide is comprised one or more of Proline (P), Glycine (G), phospho-Serine (S), phospho-Threonine (T), Arginine (R), Lysine (K), Aspartic acid (D), or Glutamic acid (E) amino acid residues. The amino acids Proline (P), Glycine (G), Arginine (R), Lysine (K), Aspartic acid (D), and Glutamic acid (E) are necessary determinants of protein structure and function. Proline and Glycine residues provide for tight turns in the 3D structure of proteins, enabling the generation of folded conformations of the polypeptide required for function. Charged amino acid sequences are often located at the surface of folded proteins and are necessary for chemical interactions mediated by the polypeptide including protein-protein interactions, protein-lipid interactions, enzyme-substrate interactions and protein-nucleic acid interactions. Thus, in some embodiments, Proline (P) and Glycine (G) Lysine (K), Aspartic acid (D), and Glutamic acid (E) rich regions proximal to the type II PDZ binding motif provide for properties necessary to the provided actions of ACT peptides. In some embodiments, the provided polypeptide comprises Proline (P) and Glycine (G) Lysine (K), Aspartic acid (D), and/or Glutamic acid (E) rich regions proximal to the type II PDZ binding motif.

Phosphorylation is the most common post-translational modification of proteins and is crucial for modulating or modifying protein structure and function. Aspects of protein structure and function modified by phosphorylation include protein conformation, protein-protein interactions, protein-lipid interactions, protein-nucleic acid interactions, channel gating, protein trafficking and protein turnover. Thus, in some embodiments the phospho-Serine (S) and/or phosphor-Threonine (T) rich sequences are necessary for modifying the function of ACT peptides, increasing or decreasing efficacy of the polypeptides in their provided actions. In some embodiments, the provided polypeptide comprise Serine (S) and/or phospho-Threonine (T) rich sequences or motifs.

In some embodiments the provided polypeptide can comprise the c-terminal sequence of human Cx43. Thus, the provided polypeptide can comprise the amino acid sequence PSSRASSRASSRPRPDDLEI (SEQ ID NO:1) or RPRPDDLEI (SEQ ID NO:2). The polypeptide can comprise 9 amino acids of the carboxy terminus of human Cx40. Thus, the polypeptide can comprise the amino acid sequence KARSDDLSV (SEQ ID NO:5).

The disclosed peptide can include one or more amino acid substitutions, for example 2-10 conservative substitutions, 2-5 conservative substitutions, 4-9 conservative substitutions, such as 2, 5 or 10 conservative substitutions.

A polypeptide can be produced to contain one or more conservative substitutions by manipulating the nucleotide sequence that encodes that polypeptide using, for example, standard procedures such as site-directed mutagenesis or PCR. Alternatively, a polypeptide can be produced to contain one or more conservative substitutions by using standard peptide synthesis methods. An alanine scan can be used to identify which amino acid residues in a protein can tolerate an amino acid substitution. In one example, the biological activity of the protein is not decreased by more than 25%, for example not more than 20%, for example not more than 10%, when an alanine, or other conservative amino acid (such as those listed below), is substituted for one or more native amino acids.

Further information about conservative substitutions can be found in, among other locations, in Ben-Bassat et al., (J. Bacterial. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988) and in standard textbooks of genetics and molecular biology.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that there are numerous amino acid and peptide analogs which can be incorporated into the disclosed compositions. The opposite stereoisomers of naturally occurring peptides are disclosed, as well as the stereoisomers of peptide analogs. These amino acids can readily be incorporated into poly-peptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994), all of which are herein incorporated by reference at least for material related to amino acid analogs).

Molecules can be produced that resemble polypeptides, but which are not connected via a natural peptide linkage. For example, linkages for amino acids or amino acid analogs can include CH₂NH—, —CH₂S—, —CH₂—CH₂—, —CH—CH— (cis and trans), COCH₂—, —CH(OH)CH₂, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J Chem. Soc Perkin Trans. I 307-314 (1982) (—CH—CH—, cis and trans); Almquist et al. J Med. Chem. 23:1392-1398 (1980) (—COCH₂—); Jennings-White et al. Tetrahedron Lett 23:2533 (1982) (—COCH₂—); Szelke et al. European Appin, EP 45665 CA (1982): 97:39405 (1982) (—CH(OH) CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. It is understood that peptide analogs can have more than one atom between the bond atoms, such as b-alanine, g-aminobutyric acid, and the like.

Amino acid analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, greater ability to cross biological barriers (e.g., gut, blood vessels, blood-brain-barrier), and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

Thus, the provided polypeptide can comprise a conservative variant of the c-terminus of an alpha Connexin (ACT). As shown in Table 1, an example of a single conservative substitution within the sequence SEQ ID NO:2 is given in the sequence SEQ ID NO:3. An example of three conservative substitutions within the sequence SEQ ID NO:2 is given in the sequence SEQ ID NO:4. Thus, the provided polypeptide can comprise the amino acid SEQ ID NO:3 or SEQ ID NO:4.

TABLE 1
ACT Polypeptide Variants

| | |
|---|---|
| RPRPDDLEI | SEQ ID NO: 2 |
| RPRPDDLEV | SEQ ID NO: 3 |
| RPRPDDVPV | SEQ ID NO: 4 |
| SSRASSRASSRPRPDDLEV | SEQ ID NO: 6 |
| RPKPDDLEI | SEQ ID NO: 7 |
| SSRASSRASSRPKPDDLEI | SEQ ID NO: 8 |
| RPKPDDLDI | SEQ ID NO: 9 |
| SSRASSRASSRPRPDDLDI | SEQ ID NO: 10 |
| SSRASTRASSRPRPDDLEI | SEQ ID NO: 11 |
| RPRPEDLEI | SEQ ID NO: 12 |
| SSRASSRASSRPRPEDLEI | SEQ ID NO: 13 |
| GDGKNSVWV | SEQ ID NO: 14 |
| GQKPPSRPSSSASKKLYV | SEQ ID NO: 15 |

It is understood that one way to define any variants, modifications, or derivatives of the disclosed genes and proteins herein is through defining the variants, modification, and derivatives in terms of sequence identity (also referred to herein as homology) to specific known sequences. Specifically disclosed are variants of the nucleic acids and polypeptides herein disclosed which have at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to the stated or known sequence. Those of skill in the art readily understand how to determine the sequence identity of two proteins or nucleic acids. For example, the sequence identity can be calculated after aligning the two sequences so that the sequence identity is at its highest level.

Another way of calculating sequence identity can be performed by published algorithms. Optimal alignment of sequences for comparison may be conducted by the local sequence identity algorithm of Smith and Waterman Adv. Appl. Math. 2: 482 (1981), by the sequence identity alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection. These references are incorporated herein by reference in their entirety for the methods of calculating sequence identity.

The same types of sequence identity can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

Thus, the provided polypeptide can comprise an amino acid sequence with at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to the c-terminus of an alpha Connexin (ACT). Thus, in one aspect, the provided polypeptide comprises an amino acid sequence with at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to SEQ ID NO:1. As an example, provided is a polypeptide (SEQ ID NO:4) having 66% sequence identity to the same stretch of 9 amino acids occurring on the carboxy-terminus of human Cx43 (SEQ ID NO:2).

In some embodiments, efficiency of cytoplasmic localization of the provided polypeptide is enhanced by cellular internalization transporter chemically linked in cis or trans with the polypeptide. Efficiency of cell internalization transporters can be enhanced further by light or co-transduction of cells with Tat-HA peptide.

Thus, the provided polypeptide can comprise a cellular internalization transporter or sequence. The cellular internalization sequence can be any internalization sequence known or newly discovered in the art, or conservative variants thereof. Non-limiting examples of cellular internalization transporters and sequences include Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynBI, Pep-7, HN-1, BGSC (Bis-Guanidinium-Spermidine-Cholesterol, and BGTC (Bis-Guanidinium-Tren-Cholesterol).

The provided polypeptide can comprise any ACT sequence (e.g, any of the ACT peptides disclosed herein) in combination with any of the herein provided cell internalization sequences. Examples of said combinations are given in Table 2. Thus, the provided polypeptide can comprise an Antennapedia sequence comprising amino acid sequence RQPKIWFPNRRKPWKK (SEQ ID NO: 38). Thus, the provided polypeptide can comprise the amino acid sequence SEQ ID NO:17, SEQ ID NO:18, SEQ ID NO:19, SEQ ID NO:20, or SEQ ID NO:21.

TABLE 2
ACT Polypeptides with Cell Interna-
lization Sequences (CIS)

| | | SEQ |
|---|---|---|
| | | ID |
| CIS/ACT | Sequence | NO: |
| Antp/ACT 2 | RQPKIWFPNRRKPWKKPSSRASSRA | 17 |
| | SSRPRPDDLEI | |
| Antp/ACT 1 | RQPKIWFPNRRKPWKKRPRPDDLEI | 18 |
| Antp/ACT 3 | RQPKIWFPNRRKPWKKRPRPDDLEV | 19 |
| Antp/ACT 4 | RQPKIWFPNRRKPWKKRPRPDDVPV | 20 |
| Antp/ACT 5 | RQPKIWFPNRRKPWKKKARSDDLSV | 21 |
| HIV-Tat/ | GRKKRRQRPPQRPRPDDLEI | 22 |
| ACT 1 | | |
| Penetratin/ | RQIKIWFQNRRMKWKKRPRPDDLEI | 23 |
| ACT 1 | | |
| Antp-3A/ | RQIAIWFQNRRMKWAARPRPDDLEI | 24 |
| ACT 1 | | |
| Tat/ACT 1 | RKKRRQRRRRPRPDDLEI | 25 |
| Buforin II/ | TRSSRAGLQFPVGRVHRLLRKRPRP | 26 |
| ACT 1 | DDLEI | |
| Transportan/ | GWTLNSAGYLLGKINKALAALAKKI | 27 |
| ACT 1 | LRPRPDDLEI | |
| MAP/ACT 1 | KLALKLALKALKAALKLARPRPDDL | 28 |
| | EI | |
| K-FGF/ACT 1 | AAVALLPAVLLALLAPRPRPDDLEI | 29 |
| Ku70/ACT 1 | VPMLKPMLKERPRPDDLEI | 30 |
| Prion/ACT 1 | ANLGYWLLALFVTMWTDVGLCKKRP | 31 |
| | KPRPRPDDLEI | |
| pVEC/ACT 1 | LLIILRRRIRKQAHAHSKRPRPDDL | 32 |
| | EI | |
| Pep-1/ACT 1 | KETWWETWWTEWSQPKKKRKVRPRP | 33 |
| | DDLEI | |
| SynB1/ACT 1 | RGGRLSYSRRRFSTSTGRRPRPDDL | 34 |
| | EI | |
| Pep-7/ACT 1 | SDLWEMMMVSLACQYRPRPDDLEI | 35 |
| HN-1/ACT 1 | TSPLNIHNGQKLRPRPDDLEI | 36 |

In some embodiments, the gap junction inhibitor is a compound having the formula (I):

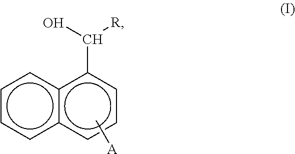

in which the quinoline ring is substituted by from one to three groups selected from halogen and trifluoromethyl (designated in the formula by “A”), and is optionally further substituted by one or more other moieties, and R is (a) NR₁R₂ in which R₁ and R₂ are independently hydrogen or C1-C4 alkyl; (b) 2-piperidyl, (c) 2-pyridyl, and (d) 5-(ethyl or vinyl)-quinuclidin-4-yl; an enantiomer of such a compound; a pharmaceutically acceptable salt of such a compound or of an enantiomer thereof; a prodrug of such a compound or of an enantiomer thereof; a metabolite of such a compound or of an enantiomer thereof; and mixtures of two or more of the foregoing

One currently known and commercially available compound of this class is mefloquine. Mefloquine is a 4-quinolinemethanol derivative with the specific chemical name of (R*,S*)-(±)-alpha-2-piperidinyl-2,8-bis(trifluoromethyl)-4-quinolinemethanol. It is a 2-aryl substituted chemical structural analog of quinine. Typically it is available and is used in the form of its hydrochloride salt. Mefloquine hydrochloride is a white to almost white crystalline compound, soluble in ethanol and slightly soluble in water.

Mefloquine has the structural formula (II):

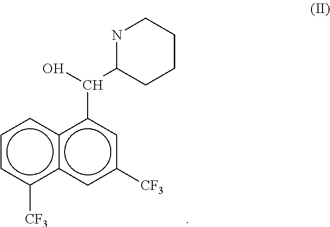

The current use of mefloquine is as an antiparasitic treatment for malaria. It is available from Roche under the trademark Lariam®. Since mefloquine has two stereocenters, there are four possible enantiomers: RS(+), SR(−), RR, and SS.

Other compounds in the class of mefloquine analogs are described in literature and patents. For example, Schmidt et al., Antimicrobial Agents and Chemotherapy 13: 1011 (1978) describes a number of such compounds (including enantiomers of mefloquine) that were screened for anti-malarial activity. Some others are disclosed, for instance in Buchman et al., J.A.C.S. 68: 2710 (1946), Rothe et al., J. Med. Chem. 11: 366 (1968), Ison et al., J. Invest. Dermatol. 52: 193 (1969), and Ohnmacht et al., J. Med. Chem. 14: 926 (1971). Schmidt et al., supra and Grethe et al., U.S. Pat. No. 3,953,453, disclose some quinuclidinyl compounds of formula (I). All these references are hereby incorporated by reference herein for the teaching of these compounds.

### Pannexin Channel Inhibitor

In some embodiments, the pannexin channel inhibitor is a pannexin channel inhibitor described in U.S. Patent Publication No. 2018/0028595, which is incorporated by reference for the teaching of these inhibitors, methods of making these inhibitors, and pharmaceutical compositions containing these inhibitors.

In some embodiments, the pannexin channel inhibitor is a peptide that mimics sequences in Panx1. For example, in some embodiments, the peptide inhibits a functional interaction between Panx1 and α1AR. In one aspect, the peptides have an additional internalization sequence, such as a TAT sequence.

In some embodiments, the peptide is a Panx1-Intracellular Loop 2 (Panx1-IL2) peptide having the amino acid sequence KYPIVEQYLK (SEQ ID NO:37). This peptide is a synthetic small-interfering peptide that mimics an important regulatory region on the intracellular loop of both human (K192-K201) and murine (K191-K200) pannexin1 proteins. In some embodiments, the Panx1-IL2 peptide has a TAT sequence and therefore can have the amino acid sequence KYPIVEQYLKYGRKKQRRR (SEQ ID NO:38).

Panx1 can be inhibited by pharmacologic inhibitors as well as inhibitors to achieve the desired results as disclosed herein. For example, in some embodiments, the pannexin-1 channel inhibitor is spironolactone. Spironolactone, sold under the brand name Aldactone® among others, is a medication that is primarily used to treat fluid build-up due to heart failure, liver scarring, or kidney disease. However, it has never been shown to be effective in treating AF or other arrhythmias.

### Pharmaceutical Formulations

Disclosed is a pharmaceutical compositions containing therapeutically effective amounts of one or more of the disclosed gap junction or pannexin channel inhibitor and a pharmaceutically acceptable carrier. Pharmaceutical carriers suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. For example, the compounds may be formulated or combined with known NSAIDs, anti-inflammatory compounds, steroids, and/or antibiotics.

The compositions contain one or more compounds provided herein. The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (See, e.g., Ansel, Introduction to Pharmaceutical Dosage Forms, 4th Edition, 1985, 126).

In one embodiment, the compositions are formulated for single dosage administration. To formulate a composition, the weight fraction of compound is dissolved, suspended, dispersed or otherwise mixed in a selected carrier at an effective concentration such that the treated condition is relieved or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro, ex vivo and in vivo systems, and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art.

Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, or in one embodiment 0.1-95%.

### Methods of Administration

The herein disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

The compositions disclosed herein may be administered prophylactically to patients or subjects who are at risk for AF. Thus, the method can further comprise identifying a subject at risk for AF prior to administration of the herein disclosed compositions.

In one embodiment, the disclosed gap junction or pannexin channel inhibitor is administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of gap junction or pannexin channel inhibitor administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

Although the gap junction or pannexin channel inhibitor may be administered once or several times a day, and the duration of the treatment may be once per day for a period of about 1, 2, 3, 4, 5, 6, 7 days or more, it is more preferably to administer either a single dose in the form of an individual dosage unit or several smaller dosage units or by multiple administration of subdivided dosages at certain intervals. For instance, a dosage unit can be administered from about 0 hours to about 1 hr, about 1 hr to about 24 hr, about 1 to about 72 hours, about 1 to about 120 hours, or about 24 hours to at least about 120 hours. Alternatively, the dosage unit can be administered from about 0.5, 1, 2, 3, 4, 5, 6, 7, 8, 9, 10, 11, 12, 13, 14, 15, 16, 17, 18, 19, 20, 21, 22, 23, 24, 30, 40, 48, 72, 96, 120 hours. Subsequent dosage units can be administered any time following the initial administration such that a therapeutic effect is achieved. The therapy with gap junction or pannexin channel inhibitor can instead include a multi-level dosing regimen wherein the gap junction or pannexin channel inhibitor is administered during two or more time periods, preferably having a combined duration of about 12 hours to about 7 days, including, 1, 2, 3, 4, or 5 days or about 15, 15, 30, 35, 40, 45, 50, 55, 60, 65, 70, 75, 80, 85, 90, 95, 100, 105, 110, 115, 120, 125, 130, 135, 140, or 144 hours or about 1 to 24 hours, about 12 to 36 hours, about 24 to 48 hours, about 36 to 60 hours, about 48 to 72 hours, about 60 to 96 hours, about 72 to 108 hours, about 96 to 120 hours, or about 108 to 136 hours. In one embodiment, the two-level gap junction or pannexin channel inhibitor dosing regimen has a combined duration of about 1 day to about 5 days; in other embodiments, the two-level gap junction or pannexin channel inhibitor dosing regimen has a combined duration of about 1 day to about 3 days.

In some embodiments, the total hourly dose of gap junction or pannexin channel inhibitor that is to be administered during the first and second time periods of the two-level progesterone or synthetic progestin dosing regimen is chosen such that a higher total dose of gap junction or pannexin channel inhibitor per hour is given during the first time period and a lower dose of gap junction or pannexin channel inhibitor per hour is given during the second time period. The duration of the individual first and second time periods of the two-level gap junction or pannexin channel inhibitor dosing regimen can vary, depending upon the health of the individual and history of the traumatic injury. Generally, the subject is administered higher total dose of gap junction or pannexin channel inhibitor per hour for at least 1, 2, 3, 4, 5, 6, 12 or 24 hours out of the 1 day to 5 day two-level gap junction or pannexin channel inhibitor dosing regimen. The length of the second time period can be adjusted accordingly, and range for example, from about 12 hrs, 24 hrs, 36 hrs, 48 hrs, 60 hrs, 72 hrs, 84 hrs, 96 hrs, 108 hrs, 120 hrs or about 12 to about 36 hrs, about 24 to about 36 hrs, about 24 to about 48 hrs, about 36 hrs to about 60 hours, about 48 hrs to about 72 hrs, about 60 hrs to about 84 hours, about 72 hrs to about 96 hrs, or about 108 hrs to about 120 hrs. Thus, for example, where the two-level gap junction or pannexin channel inhibitor dosing regimen has a combined duration of 3 days, the higher total doses of gap junction or pannexin channel inhibitor could be administered for the first hour, and the lower total hourly dose of gap junction or pannexin channel inhibitor could be administered for hours 2 to 72.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Vascular Endothelial Growth Factor Promotes Atrial Arrhythmias by Inducing Acute Intercalated Disk Remodeling

Introduction

Atrial fibrillation (AF) is the most common cardiac arrhythmia, affecting 2-3% of the US population (Zoni-Berisso M, et al. Clin Epidemiol. 2014 6:213-20). Inflammation, vascular leak, and associated tissue edema are common sequelae of pathologies associated with AF (Weis S M. Curr Opin Hematol. 2008 15:243-9; Li J, et al. Heart rhythm. 2010 7:438-44; Ogi H, et al. Circulation journal. 2010 74:1815-21; Scridon A, et al. Europace. 2012 14:948-53; Seko Y, et al. Jpn Heart J. 2000 41:27-32; Gramley F, et al. Cardiovasc Pathol. 2010 19:102-11; Chung N A, et al. Stroke. 2002 33:2187-91) and are emerging as proarrhythmic factors. Inflammatory signaling involving cytokines, such as VEGF, and mediated by VEGF receptor 2 compromise the vascular barrier function, and increase vascular leak (Sukriti S, et al. Pulm Circ. 2014 4:535-51). Multiple studies in early stage AF patients (lone/paroxysmal AF) report elevated levels of VEGF (89-560 pg/ml) (Li J, et al. Heart rhythm. 2010 7:438-44; Ogi H, et al. Circulation journal. 2010 74:1815-21; Scridon A, et al. Europace. 2012 14:948-53; Seko Y, et al. Jpn Heart J. 2000 41:27-320; Chung N A, et al. Stroke. 2002 33:2187-91) and VEGF receptor 2 (Gramley F, et al. Cardiovasc Pathol. 2010 19:102-11). Likewise, elevated levels of vascular leak-inducing cytokines predict AF recurrence following ablation (Kimura T, et al. Heart Lung Circ. 2014 23:636-43). Although vascular leak is appreciated as a chronic contributor to adverse remodeling and cardiovascular disease (Bertoluci M C, et al. World J Diabetes. 2015 6:679-92; de Zeeuw D, et al. J Am Soc Nephrol. 2006 17:2100-5; Montezano A C, et al. Can J Cardiol. 2015 31:631-641), its acute contribution to arrhythmogenesis has yet to be explored. Myocardial edema, a direct consequence of vascular leak, is linked to arrhythmias in multiple pathologies, including AF (Amano Y, et al. ScientificWorldJournal. 2012 2012:194069; Boyle A, et al. Journal of cardiac failure. 2007 13:133-6; White S K, et al. JACC Cardiovasc Interv. 2015 8:178-88; Zia M I, et al. The American journal of cardiology. 2014 113:607-12; Migliore F, et al. Heart rhythm. 2015). Likewise, cardiac edema has been linked to AF recurrence following ablation (Neilan T G, et al. JACC Cardiovasc Imaging. 2014 7:1-11; Arujuna A, et al. Circulation Arrhythmia and electrophysiology. 2012 5:691-70).

There is evidence that interstitial edema may acutely (within minutes) elevate arrhythmia susceptibility (George S A, et al. Front Physiol. 2017 8:334; Veeraraghavan R, et al. Pflugers Arch. 2015 467:2093-2105; Veeraraghavan R, et al. Pflugers Arch. 2016 468:1651-61; Veeraraghavan R, et al. Am J Physiol Heart Circ Physiol. 2012 302(1):H278-86). The proarrhythmic impact of edema resulted from disruption of cardiac sodium channel (Naᵥ1.5)-rich intercalated disk (ID) nanodomains and consequent slowing of action potential propagation (Veeraraghavan R, et al. Pflugers Arch. 2015 467:2093-2105; Veeraraghavan R, et al. Pflugers Arch. 2016 468:1651-61; Veeraraghavan R, et al. Am J Physiol Heart Circ Physiol. 2012 302(1):H278-86; Veeraraghavan R, et al. Elife. 2018 7). Interestingly, similar disruption of ID nanodomains has been identified in AF patients (Raisch T B, et al. Front Physiol. 2018). Therefore, VEGF (at clinically-relevant levels) may acutely promote atrial arrhythmias by disrupting ID nanodomains and slowing atrial conduction. Disclosed in this Example is structural and functional evidence, from the nanoscale to the in vivo level, demonstrating that this mechanism can promote atrial arrhythmias. Also disclosed is a new form of tissue remodeling involving the dynamic reorganization of Naᵥ1.5 within the ID occurring in the aftermath of acute exposure to VEGF, resulting in the dispersal of channels from dense clusters located within nanodomains.

Methods

All animal procedures were approved by Institutional Animal Care and Use Committee at The Ohio State University and performed in accordance with the Guide for the Care and Use of Laboratory Animals published by the U.S. National Institutes of Health (NIH Publication No. 85-23, revised 2011).

Langendorff Preparation, Tissue Collection: Male C57/BL6 mice (30 grams, 6-18 weeks) were anesthetized with 5% isoflurane mixed with 100% oxygen (1 l/min). After loss of consciousness, anesthesia was maintained with 3-5% isoflurane mixed with 100% oxygen (1 l/min). Once the animal was stably in a surgical plane of anesthesia, the heart was excised, leading to euthanasia by exsanguination. The isolated hearts were prepared in one of the following three ways.

Langendorff preparations: For optical mapping and ex vivo electrocardiography (ECG) studies, hearts were perfused (at 40-55 mm Hg) in a Langendorff configuration with oxygenated, modified Tyrode's solution (containing, in mM: NaCl 140, KCl 5.4, MgCl₂ 0.5, dextrose 5.6, HEPES 10; pH adjusted to 7.4) at 37° C. as previously described (Veeraraghavan R, et al. Pflugers Arch. 2015 467:2093-2105; Veeraraghavan R, et al. Elife. 2018 7; Radwanski P B, et al. Cardiovascular research. 2015 106:143-52; Radwanski P B, et al. Heart rhythm. 2010 7:1428-35; Veeraraghavan R and Poelzing S. Cardiovascular research. 2008 77:749-56.

Cryopreservation: Hearts were embedded in optimal cutting temperature compound and frozen using liquid nitrogen for cryosectioning and fluorescent immunolabeling as in previous studies (Veeraraghavan R, et al. Pflugers Arch. 2015 467:2093-2105; Veeraraghavan R, et al. Pflugers Arch. 2016 468:1651-61; Veeraraghavan R, et al. Elife. 2018 7; Veeraraghavan R and Gourdie R. Molecular biology of the cell. 2016 27:3583-3590). These samples were used for light microscopy experiments as described below.

Fixation for Transmission Electron Microscopy (TEM): Atria were dissected and fixed overnight in 2% glutaraldehyde at 4° C. for resin embedding and ultramicrotomy as previously described (Veeraraghavan R, et al. Pflugers Arch. 2015 467:2093-2105; Veeraraghavan R, et al. Elife. 2018 7).

FITC-dextran extravasation: Langendorff-perfused mouse hearts were perfused for 60 minutes with Tyrode's solution with or without VEGF (500 pg/ml) and FITC-dextran (10 mg/ml) was added to the final 10 ml of perfusate. Perfused hearts were then cryopreserved as described above and extravasated FITC-dextran levels assessed by confocal microscopy of cryosections.

Optical Mapping and Volume-conducted Electrocardiography (ECG): Optical voltage mapping was performed using the voltage sensitive dye, di-4-ANEPPS (15 μM; ThermoFisher Scientific, Grand Island, N.Y.), as previously described (Veeraraghavan R, et al. Pflugers Arch. 2015 467:2093-2105; Veeraraghavan R, et al. Pflugers Arch. 2016 468:1651-61; Veeraraghavan R and Poelzing S. Cardiovascular research. 2008 77:749-56), in order to quantify conduction velocity. Motion was suppressed by adding blebbistatin (10 μM) to the perfusate. Preparations were excited by 510 nm light and fluorescent signals passed through a 610 nm longpass filter (Newport, Irvine, Calif.) and recorded at 1000 frames/sec using a MiCAM Ultima-L CMOS camera (SciMedia, Costa Mesa, Calif.). Activation time was defined as the time of the maximum first derivative of the AP (Girouard S D, et al. J Cardiovasc Electrophysiol. 1996 7:1024-38), and activation times were fitted to a parabolic surface (Bayly P V, et al. IEEE Trans Biomed Eng. 1998 45:563-71). Gradient vectors evaluated along this surface were averaged along the fast axis of propagation (±15°) to quantify CV. Hearts were paced epicardially from the left atrium at a cycle length of 100 ms with 1 ms current pulses at 1.5 times the pacing threshold for all CV measurements. A volume-conducted ECG was collected concurrently using silver chloride electrodes placed in the bath and digitized at 1 kHz. Atrial arrhythmia inducibility was assessed by 10 s of burst pacing at cycle lengths of 50, 40, and 30 ms as previously described (Greer-Short A, et al. Heart rhythm. 2020 17:503-511; Aschar-Sobbi R, et al. Nat Commun. 2015 6:6018).

In subsets of experiments, vascular endothelial growth factor A (VEGF; Sigma SRP4364) was added to the perfusate at 100 (low) and 500 pg/ml (high). These concentrations were selected based on VEGF levels observed in human AF patients (89-560 pg/ml) (Li J, et al. Heart rhythm. 2010 7:438-44; Ogi H, et al. Circulation journal. 2010 74:1815-21; Scridon A, et al. Europace. 2012 14:948-53; Seko Y, et al. Jpn Heart J. 2000 41:27-32; Chung N A, et al. Stroke. 2002 33:2187-91). Measurements were made following 30 minutes of treatment.

In vivo ECG: Continuous ECG recordings (PL3504 PowerLab 4/35, ADInstruments) were obtained from mice anesthetized with isoflurane (1-1.5%) as previously described (Koleske M, et al. The Journal of general physiology. 2018). Briefly, after baseline recording (5 min.), animals received either intraperitoneal VEGF (10 or 50 ng/kg; Sigma) or vehicle (PBS). After an additional 20 min, animals were injected intraperitoneally with epinephrine (1.5 mg/kg; Sigma) and caffeine (120 mg/kg; Sigma) challenge and ECG recording continued for 40 minutes. ECG recordings were analyzed using the LabChart 8 software (ADInstruments).

Primary Antibodies: The following primary antibodies were used for Western immunoblotting and fluorescence microscopy studies: connexin43 (Cx43; rabbit polyclonal; Sigma C6219); connexin40 (Cx40; rabbit polyclonal; ThermoFisher Scientific 36-4900); N-cadherin (N-cad; mouse monoclonal; BD Biosciences 610920); cardiac isoform of the voltage-gated sodium channel (Naᵥ1.5; rabbit polyclonal; custom antibody (Veeraraghavan R, et al. Elife. 2018 7)); and the sodium channel β subunit (β1; rabbit polyclonal; custom antibody (Veeraraghavan R, et al. Elife. 2018 7))

Western Immunoblotting: Whole cell lysates of mouse hearts frozen using liquid nitrogen were prepared as previously described (Veeraraghavan R, et al. Elife. 2018 7; Koleske M, et al. The Journal of general physiology. 2018; Struckman H L, et al. Microsc Microanal. 2020:1-9). These were electrophoresed on 4-15% TGX Stain-free gels (BioRad, Hercules, Calif.) before being transferred onto a nitrocellulose membrane. The membranes were probed with primary antibodies against Cx43, Cx40, Naᵥ1.5 and 131 as well as mouse monoclonal antibody against GAPDH (loading control; Fitzgerald Industries, Acton, Mass.), followed by goat anti-rabbit and goat anti-mouse HRP-conjugated secondary antibodies (Promega, Madison, Wis.). Signals were detected by chemiluminescence using SuperSignal West Femto Extended Duration Substrate (ThermoFisher Scientific, Grand Island, N.Y.) and imaged using a Chemidoc MP imager (BioRad, Hercules, Calif.).

Fluorescent Immunolabeling: Immuno-fluorescent labeling of cryosections (5 μm thickness) of fresh-frozen myocardium was performed, as previously described (Veeraraghavan R, et al. Pflugers Arch. 2015 467:2093-2105; Veeraraghavan R, et al. Elife. 2018 7; Koleske M, et al. The Journal of general physiology. 2018; Radwanski P B, et al. JACC: Basic to Translational Science. 2016 1:251-266). Briefly, cryosections were fixed with paraformaldehyde (2%, 5 minutes at room temperature), permeabilized with Triton X-100 (0.2% in PBS for 15 minutes at room temperature) and treated with blocking agent (1% BSA, 0.1% triton in PBS for 2 hours at room temperature) prior to labeling with primary antibodies (overnight at 4° C.). Samples were then washed in PBS (3×5 minutes in PBS at room temperature) prior to labeling with secondary antibodies.

For confocal microscopy, samples were then labeled with goat anti-mouse and goat anti-rabbit secondary antibodies conjugated to Alexa 405, Alexa 488, Alexa 568 and Alexa 647 were used (1:8000; ThermoFisher Scientific, Grand Island, N.Y.). Simultaneous labeling with two rabbit or mouse primary antibodies was accomplished by direct fluorophore conjugation of primary antibodies (Zenon labeling kits, ThermoFisher Scientific, Grand Island, N.Y.). Samples were then washed in PBS (3×5 minutes in PBS at room temperature) and mounted in ProLong Gold (Invitrogen, Rockford, Ill.). For STimulated Emission Depletion (STED) microscopy, samples were prepared similar to confocal microscopy but labeled with Alexa 594 and Atto 647N fluorophores. For STochastic Optical Reconstruction Microscopy (STORM), samples were labeled with Alexa 647 and Biotium CF 568 fluorophores. STORM samples were then washed in PBS (3×5 minutes in PBS at room temperature) and optically cleared using Scale U2 buffer (48 hours at 4° C.) prior to imaging (Veeraraghavan R, et al. Pflugers Arch. 2016 468:1651-61; Veeraraghavan R, et al. Elife. 2018 7; Veeraraghavan R and Gourdie R. Molecular biology of the cell. 2016 27:3583-3590).

Transmission Electron Microscopy (TEM): TEM images of the ID, particularly gap junctions (GJs) and mechanical junctions (MJs), were obtained at 60,000× magnification on a FEI Tecnai G2 Spirit electron microscope. Intermembrane distance at various ID sites was quantified using ImageJ (NIH), as previously described (Veeraraghavan R, et al. Pflugers Arch. 2015 467:2093-2105; Veeraraghavan R, et al. Elife. 2018 7).

Sub-diffraction Confocal Imaging (sDCI): Confocal imaging was performed using an A1R-HD laser scanning confocal microscope equipped with four solid-state lasers (405 nm, 488 nm, 560 nm, 640 nm, 30 mW each), a 63×/1.4 numerical aperture oil immersion objective, two GaAsP detectors, and two high sensitivity photomultiplier tube detectors (Nikon, Melville, N.Y.). Individual fluorophores were imaged sequentially with the excitation wavelength switching at the end of each frame. Images were collected as z-stacks with fluorophores images sequentially (line-wise) to achieve optimal spectral separation. Sub-diffraction structural information (130 nm resolution) was recovered by imaging with a 12.8 μm pinhole (0.3 Airy units) with spatial oversampling (4× Nyquist sampling) and applying 3D deconvolution, as previously described (Lam F, et al. Methods. 2017 115:17-27).

STimulated Emission Depletion (STED) Microscopy: Samples were imaged using a time-gated STED 3× system (Leica, Buffalo Grove, Ill.) based on a TCS SP8 laser scanning confocal microscope and equipped with STED modules, a pulsed white-light laser (470-670 nm; 80 MHz pulse rate), a Plan Apochromat STED WHITE 100×/1.4 numerical aperture oil immersion objective, HyD hybrid detectors, and three STED depletion lasers (775 nm, 660 nm, 592 nm). Depletion beam was applied in the classical vortex donut configuration to achieve the best lateral resolution (25 nm) as well as in a z-donut configuration to achieve the best axial resolution (50 nm). Time gating of light collection (1.5-3.5 ns following each laser pulse) was also applied to aid in achieving optimal resolution. Images were collected as z-stacks with fluorophores images sequentially (line-wise) and subjected to 3D deconvolution. These images were analyzed using object-based segmentation in 3D (OBS3D), as previously described (Veeraraghavan R, et al. Pflugers Arch. 2015 467:2093-2105; Veeraraghavan R, et al. Pflugers Arch. 2016 468:1651-61).

Single Molecule Localization: STORM imaging was performed using a Vutara 352 microscope (Bruker Nano Surfaces, Middleton, Wis.) equipped with biplane 3D detection, and fast sCMOS imaging achieving 20 nm lateral and 50 nm axial resolution, as previously described (Veeraraghavan R, et al. Elife. 2018 7; Veeraraghavan R and Gourdie R. Molecular biology of the cell. 2016 27:3583-3590; Struckman H L, et al. Microsc Microanal. 2020:1-9; Bonilla I M, et al. Sci Rep. 2019 9:10179). Individual fluorophore molecules were localized with a precision of 10 nm. The two-color channels were precisely registered using localized positions of several TetraSpeck Fluorescent Microspheres (ThermoFisher Scientific, Carlsbad, Calif.) scattered throughout the field of view, with the procedure being repeated at the start of each imaging session. Protein clustering and spatial organization were quantitatively assessed from single molecule localization data using STORM-RLA, a machine learning-based cluster analysis approach, as previously described (Veeraraghavan R and Gourdie R. Molecular biology of the cell. 2016 27:3583-3590).

Statistical Analysis: All data which passed the Shaprio-Wilk test for normality were treated as follows. The Wilcoxon signed rank test or a single factor ANOVA was used for single comparisons. For multiple comparisons, the Šidák correction was applied. Fisher's exact test was used to test differences in nominal data. For non-normal data, a Friedman rank sum test or Kruskal-Wallis 1-way analysis of variance for paired and unpaired data was applied. A p<0.05 was considered statistically significant. All values are reported as mean±standard error unless otherwise noted.

Results

Multiple studies in early stage AF patients (lone/paroxysmal AF) report elevated levels of VEGF (89-560 pg/ml) (Li J, et al. Heart rhythm. 2010 7:438-44; Ogi H, et al. Circulation journal. 2010 74:1815-21; Scridon A, et al. Europace. 2012 14:948-53; Seko Y, et al. Jpn Heart J. 2000 41:27-32; Chung N A, et al. Stroke. 2002 33:2187-91) and VEGF receptor 2 (Gramley F, et al. Cardiovasc Pathol. 2010 19:102-11). In order to assess the acute impact of VEGF on AF susceptibility, the structural impacts of treating Langendorff-perfused WT mouse hearts with clinically relevant levels of VEGF (low: 100 pg/ml and high: 500 pg/ml) for 30 minutes was assessed.

VEGF Treatment Acutely Enhances Vascular Leak

First, extravasation of FITC-dextran as a measure of vascular leak was quantified. Levels of FITC-dextran extravasated into VEGF-treated (500 pg/ml) hearts was doubled relative to vehicle controls (201±7% vs. 100±9%, p<0.05, n=3 hearts/group). These data are consistent with acute enhancement of vascular leak by VEGF.

Atrial Conduction is Slowed Following Acute VEGF Treatment

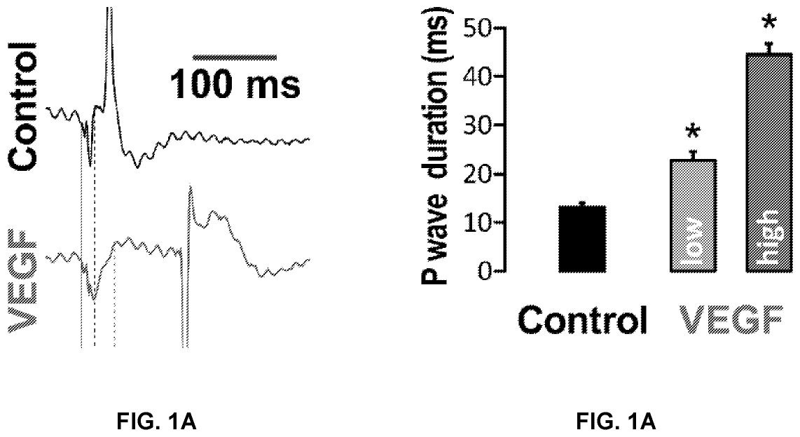

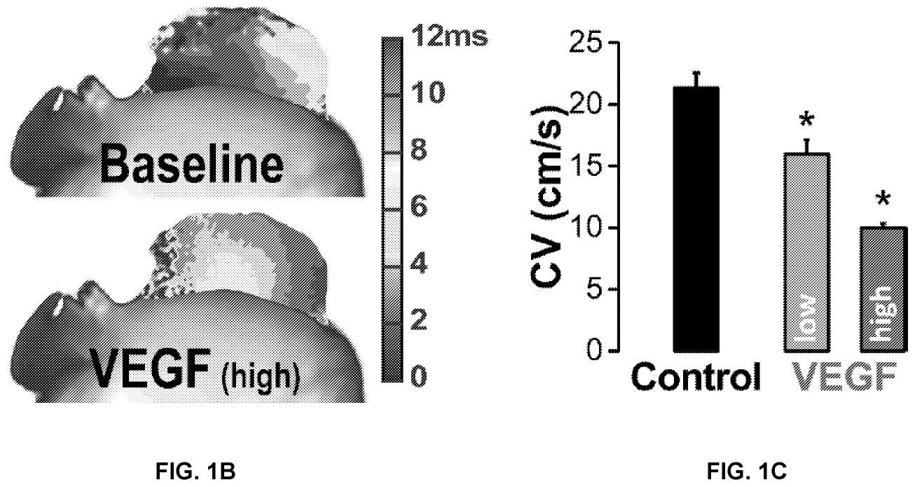

To examine the functional impacts of VEGF-induced ID remodeling, volume-conducted electrocardiograms (ECG) were recorded from Langendorff-perfused mouse hearts. A representative ECG trace in FIG. 1A shows P-wave prolongation following 30 minutes of VEGF perfusion relative to untreated control. Summary data revealed significant P-wave prolongation by VEGF (FIG. 1B). These data point to possible slowing of atrial conduction following VEGF treatment. Next, atrial conduction velocity was directly assessed using optical voltage mapping. Representative optical isochrone maps of activation in FIG. 1C demonstrate increased conduction delay in VEGF treated hearts compared to untreated controls. Overall, VEGF significantly and dose-dependently decreased atrial conduction velocity (FIG. 1D).

VEGF-Treated Hearts are Susceptible to Atrial Arrhythmias

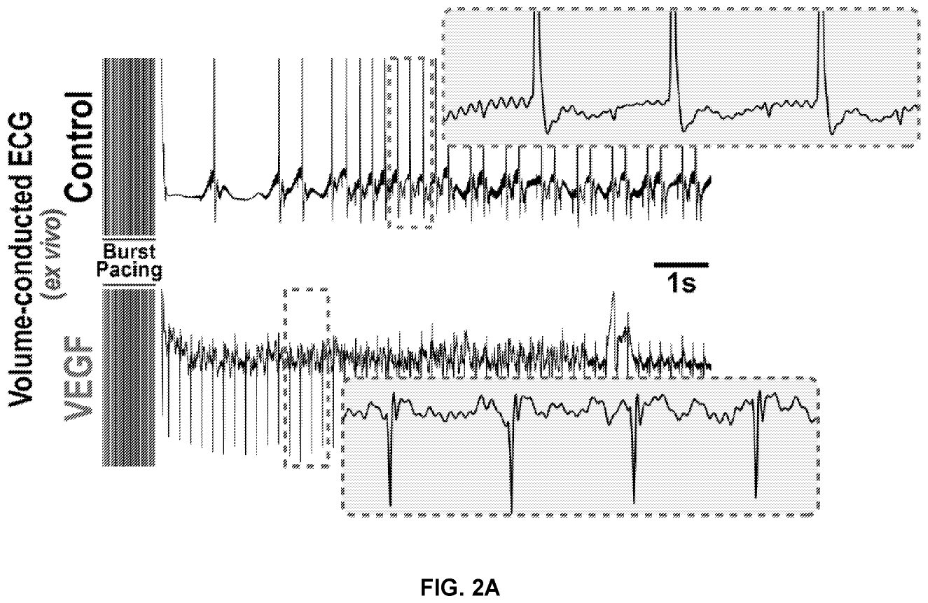

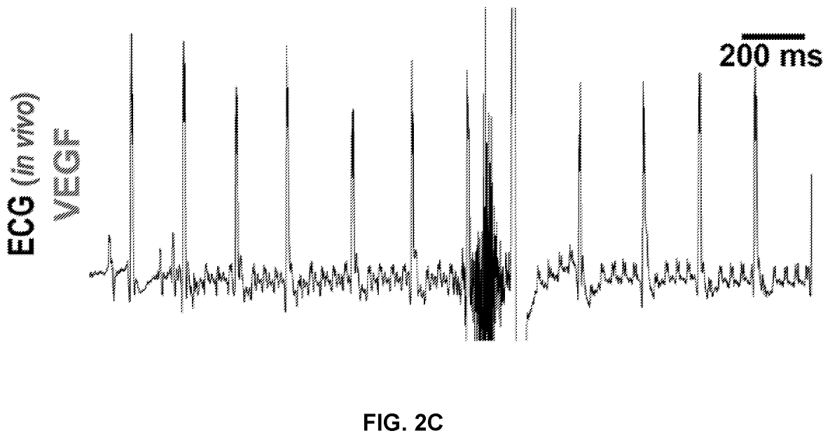

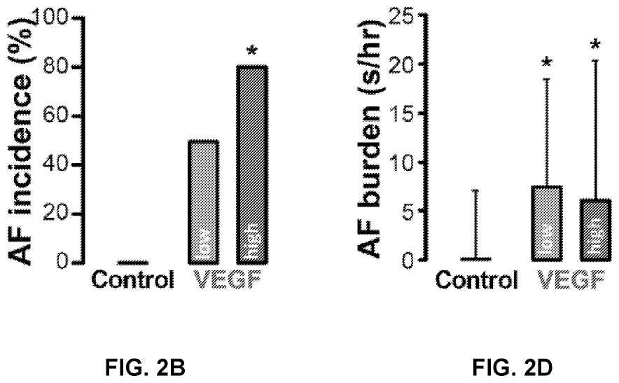

Conduction slowing is a well-established substrate for cardiac arrhythmias in general (Kleber A G and Rudy Y. Physiological reviews. 2004 84:431-88; Kleber A G. J Cardiovasc Electrophysiol. 1999 10:1025-7; Radwanski P B, et al: An Emerging View. Front Physiol. 2018 9:1228), and AF in particular (Zheng Y, et al. Clin Physiol Funct Imaging. 2017 37:596-601; Lalani G G, et al. J Am Coll Cardiol. 2012 59:595-606). Therefore, the acute effects of VEGF-induced conduction slowing on AF risk was assessed. A representative volume-conducted ECG trace in FIG. 2A (top) illustrates resumption of sinus rhythm following burst pacing. In contrast, an atrial arrhythmia is apparent on the trace from a VEGF-treated heart (FIG. 2A, bottom). Overall, VEGF increased the incidence of burst pacing-induced atrial arrhythmias in dose-dependent fashion (FIG. 2A, 2B).

Next, the acute impact of VEGF on atrial arrhythmia risk was assessed in vivo. Promotion of arrhythmic triggers via caffeine and epinephrine challenge elicited atrial arrhythmias in VEGF-treated mice but not in untreated controls (FIG. 2C, 2D). Taken together, these data suggest that conduction slowing increases the risk of atrial arrhythmias.

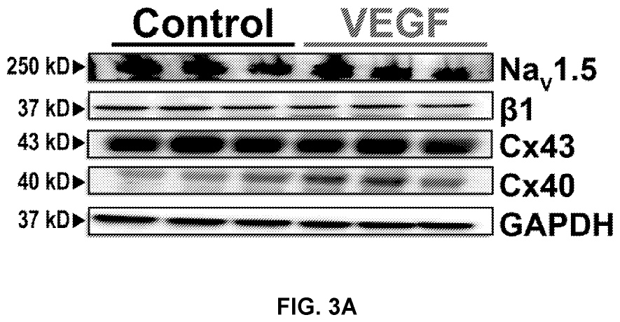

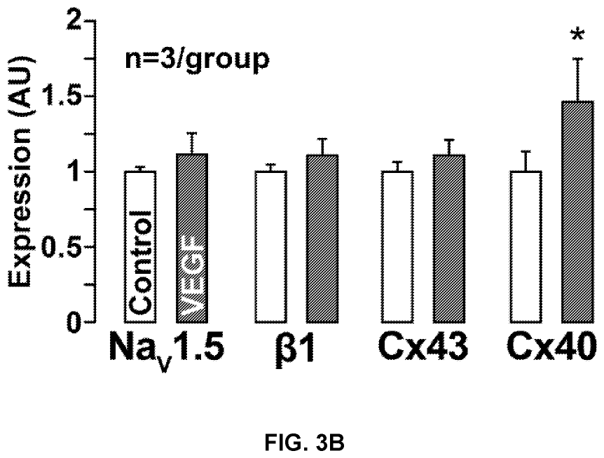

VEGF does not Acutely Alter Expression of Key ID Proteins In order to determine the structural basis of VEGF-induced atrial arrhythmias, the expression of key ID proteins was assessed. Western immunoblotting revealed no significant difference in the levels of Na⁺ channel subunits (Naᵥ1.5, β1), the gap junction protein Cx43, or the mechanical junction protein, N-cad between VEGF-treated (high dose) hearts and untreated controls (FIG. 3). Expression of the gap junction protein Cx40 was slightly elevated in VEGF-treated hearts. Increased Cx40 expression could enhance GJ coupling, although the small change observed is unlikely to have appreciable functional impact. In any case, changes in ID protein expression cannot explain VEGF-induced conduction slowing and proarrhythmia.

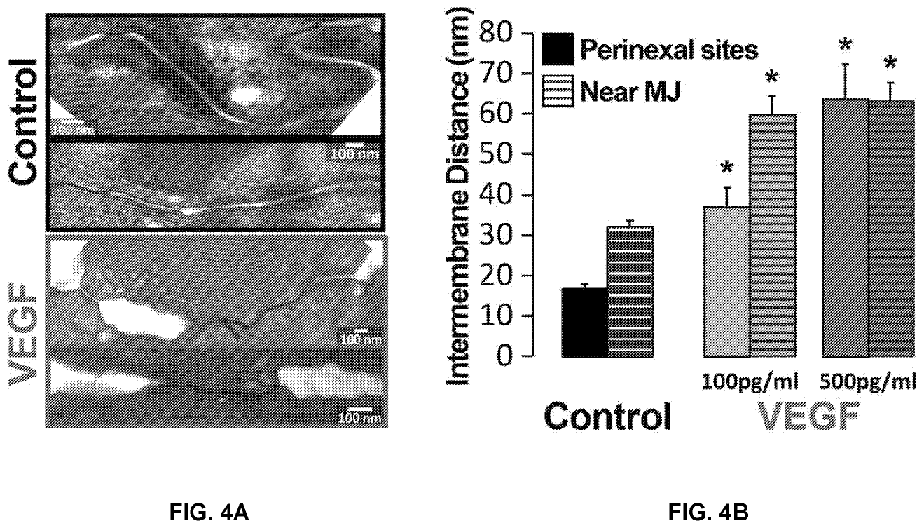

ID Structural Remodeling Following Acute VEGF Insult Previous studies link cardiac interstitial edema to ultrastructural remodeling within the ID, specifically, increased intermembrane distance near GJ. Similar changes have also been reported in AF patients (Raisch T B, et al. Front Physiol. 2018). Therefore, transmission electron microscopy (TEM) was performed to assess the acute effects of VEGF on ID structure. Representative TEM images show narrow intermembrane spacing at GJ- and MJ-adjacent sites in untreated control hearts, and marked widening at these sites following VEGF treatment (FIG. 4A). Overall, both low and high doses of VEGF significantly increased intermembrane distances at GJ- and MJ-adjacent sites compared to untreated controls (FIG. 4B). The swelling occurred in dose-dependent fashion at GJ-adjacent perinexi but not near MJ.

ID Proteins Undergo Reorganization Following Acute VEGF Treatment

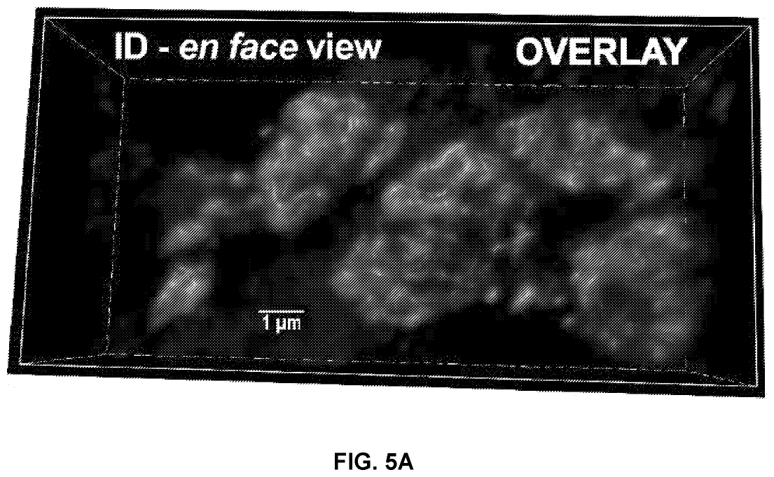

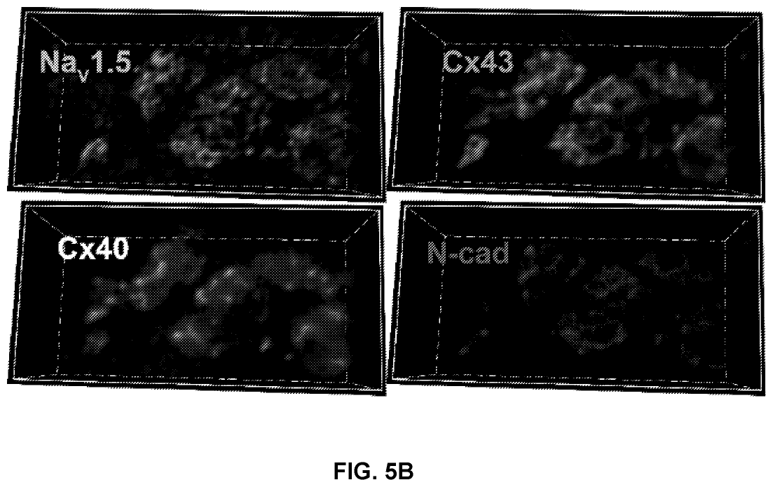

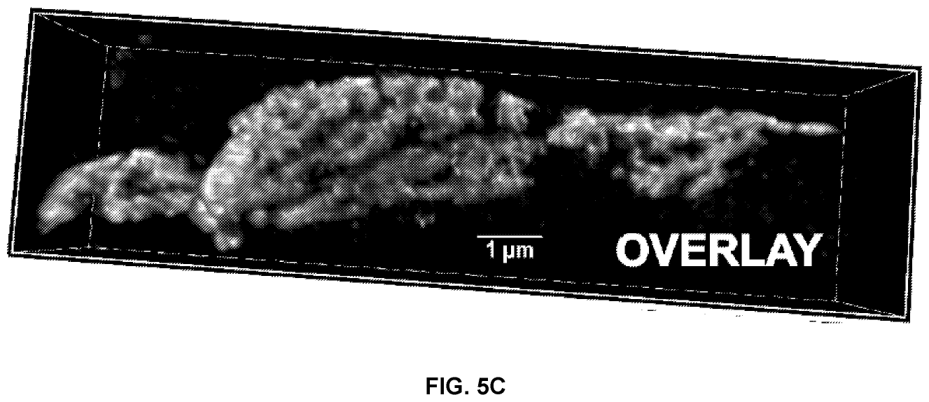

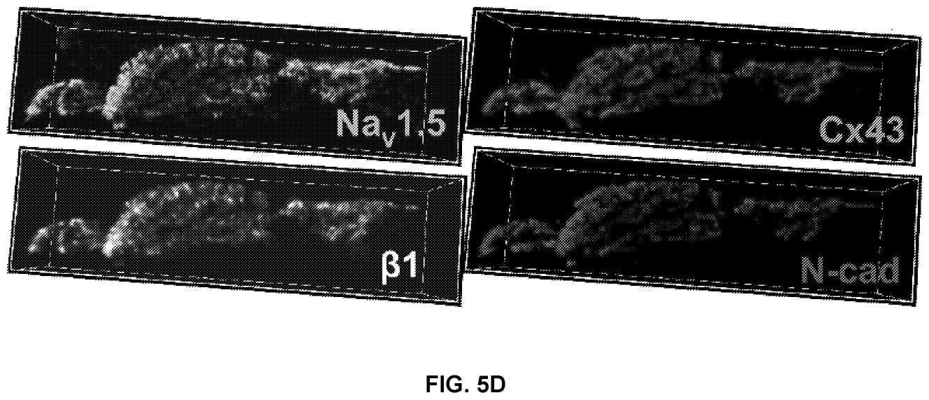

Next, super-resolution microscopy studies were performed to assess VEGF's effects on ID molecular organization. As a first step, sDC imaging (130 nm resolution) was used to examine the overall layout of key proteins within the murine atrial ID. Although lacking the resolution of other super-resolution imaging methods such as STED and STORM, sDCI offers greater capability for multicolor imaging. Therefore, sDCI was used to examine the organization of sodium channel α (NaV1.5) and β (β1) subunits relative to GJ (Cx40, Cx43) and MJ (N-cad) proteins (FIG. 5).

Both connexin isoforms predominantly expressed in the atria, Cx40 and Cx43, displayed similar patterns of localization, wherein they were organized into dense punctate clusters throughout the ID (FIG. 5A, 5B). This similarity in their patterns of distribution suggested that either isoform could be used as a marker for atrial GJs. N-cad was observed to be densest at ID sites located in between connexin clusters with very little co-localization. These results are consistent with the enrichment of GJ and MJ within interplicate and plicate ID regions respectively.

Representative sDCI images (FIG. 5C, 5D) illustrate an ID in en face orientation from a murine atrial section labeled for Naᵥ1.5, β1, Cx43 and N-cad. Naᵥ1.5 was distributed extensively throughout the ID, largely organized in the form of dense clusters. Naᵥ1.5 clusters could be identified in close proximity to Cx43 clusters as well as at N-cad-rich sites. In contrast, β1 was preferentially distributed to Cx43-adjacent sites in comparison to N-cad adjacent sites, and co-distributed with Naᵥ1.5 at these locations.

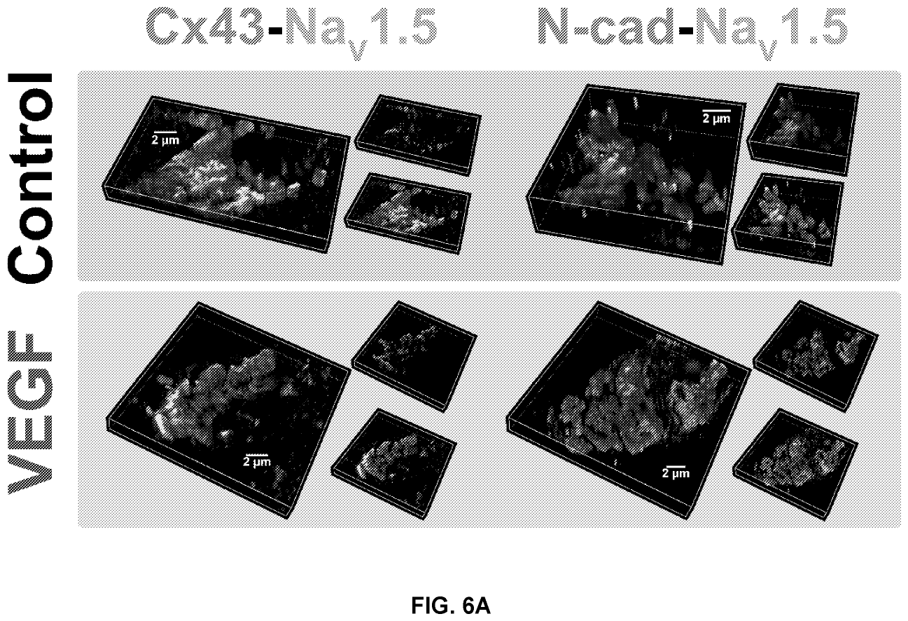

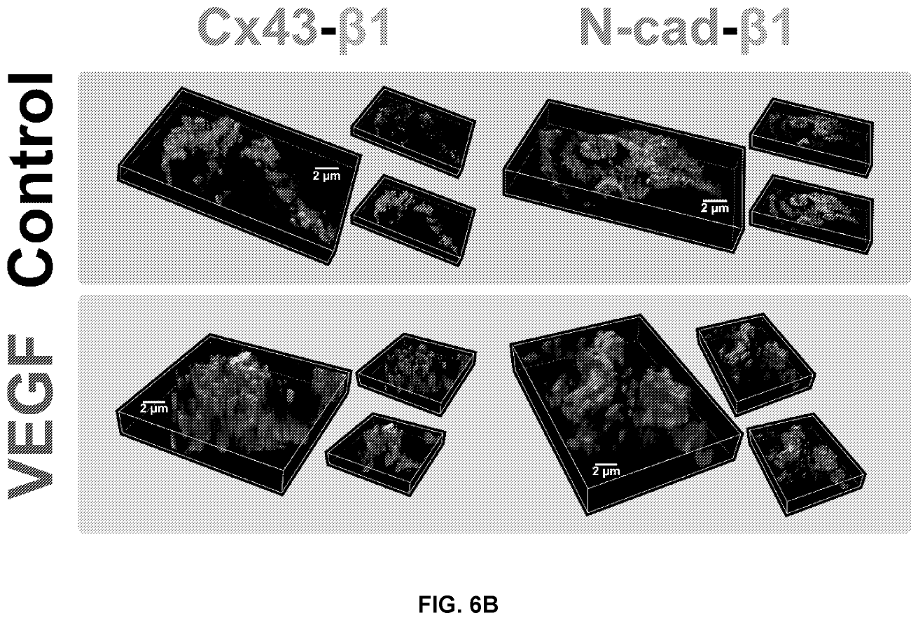

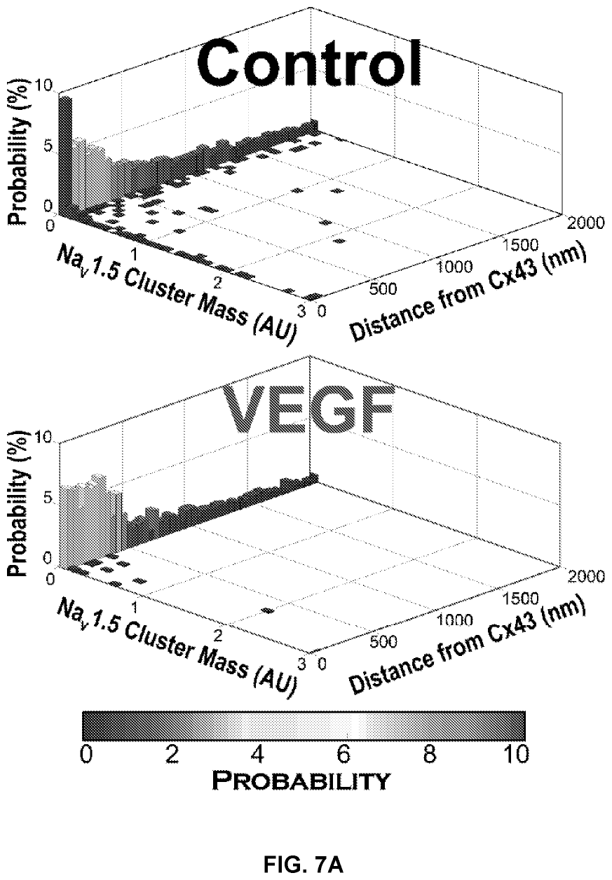

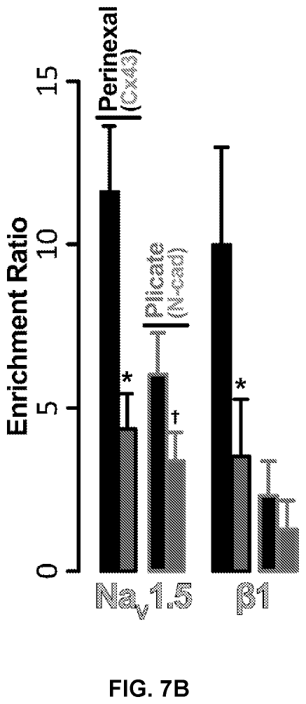

Having established the overall layout of Na⁺ channel components within the atrial ID, higher resolution techniques were used to assess the effects of VEGF-induced vascular leak on their localization. Three dimensional en face views of IDs obtained by STED microscopy (25 nm resolution) are presented in FIG. 6. In untreated control hearts, STED revealed extensive clustering of Naᵥ1.5 throughout the ID, particularly in close proximity to Cx43 clusters and at N-cad-rich sites (FIG. 6A, top). In VEGF-treated hearts, Naᵥ1.5 clusters appeared fragmented, were located further from Cx43 clusters, and co-distributed less with N-cad (FIG. 6A, bottom). Similar to Naᵥ1.5, β1 was also organized into clusters, and was found in close proximity to Cx43 clusters (FIG. 6B, top). However, unlike Naᵥ1.5, β1 displayed very little co-distribution with N-cad. In VEGF-treated hearts, β1 clusters appeared more diffuse and were distributed farther away from Cx43 clusters (FIG. 6B, bottom). Quantitative analysis by object-based segmentation was used to calculate Naᵥ1.5 and β1 signal enrichment ratio, defined as the ratio of Naᵥ1.5/β1 immunosignal mass (volume x normalized intensity) at sites near (<100 nm away) Cx43 and N-cad vs. the signal mass at other ID sites. Overall, we observed revealed significant enrichment of Naᵥ1.5 immunosignal near (<100 nm) Cx43 and N-cad, and β1 near Cx43 (FIG. 7). VEGF-treatment significantly decreased Naᵥ1.5 and β1 enrichment ratio near Cx43, while Naᵥ1.5 also trended towards a decrease at N-cad-rich sites. These results suggest that VEGF-induced vascular leak may induce acute reorganization of Naᵥ1.5 and β1 within the ID.

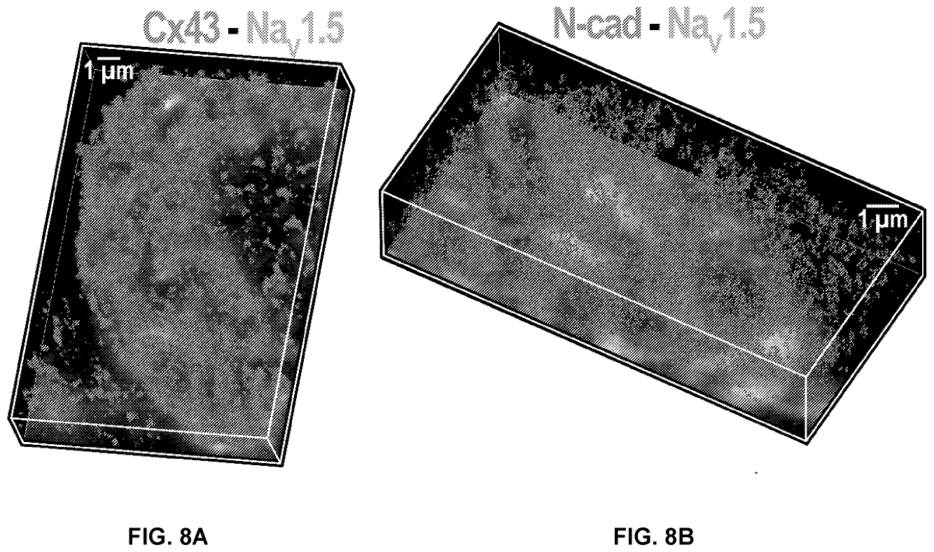

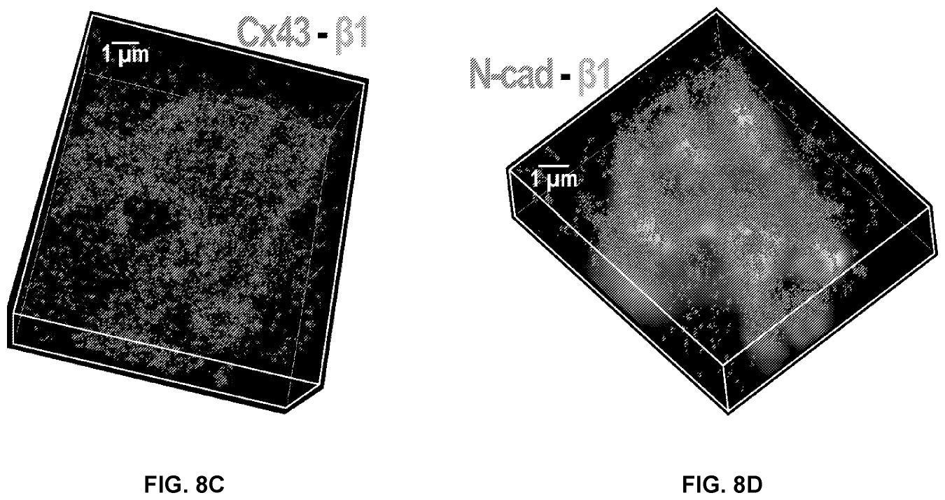

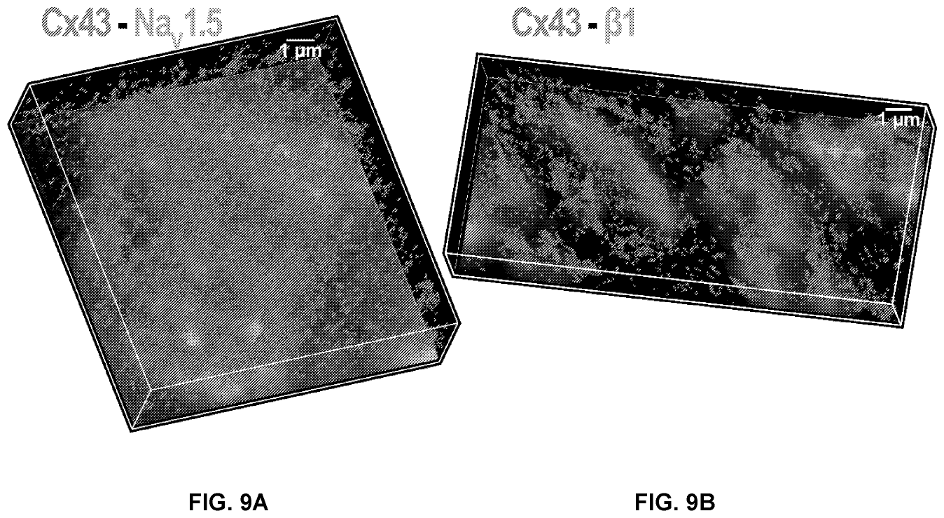

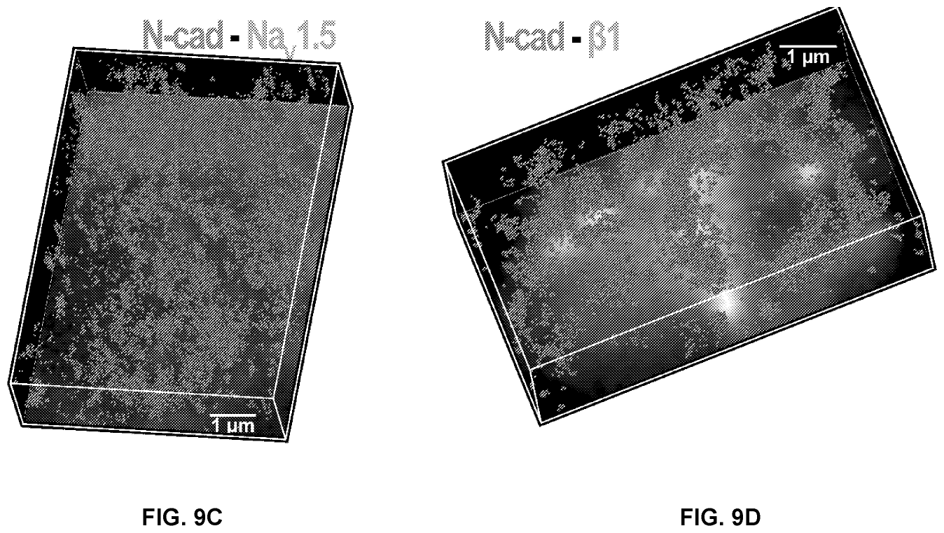

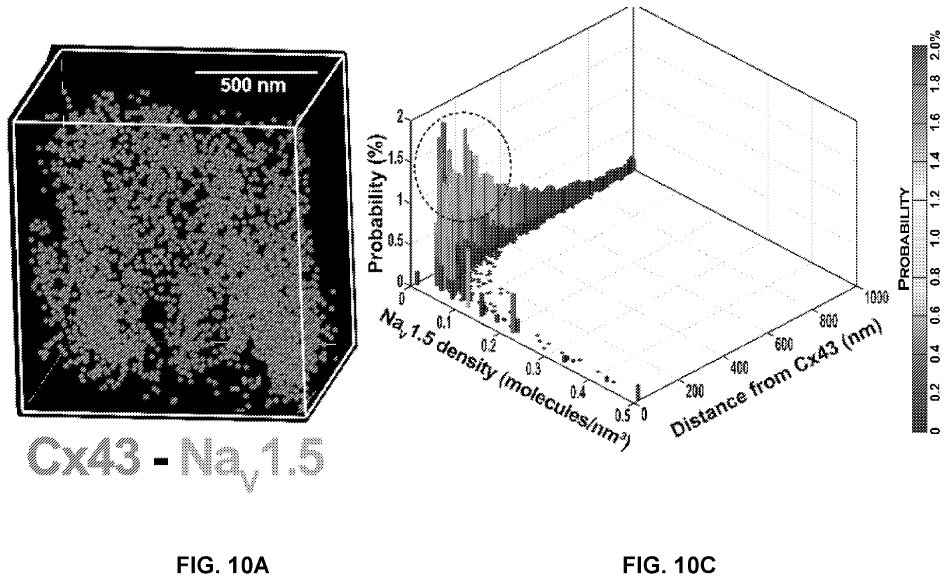

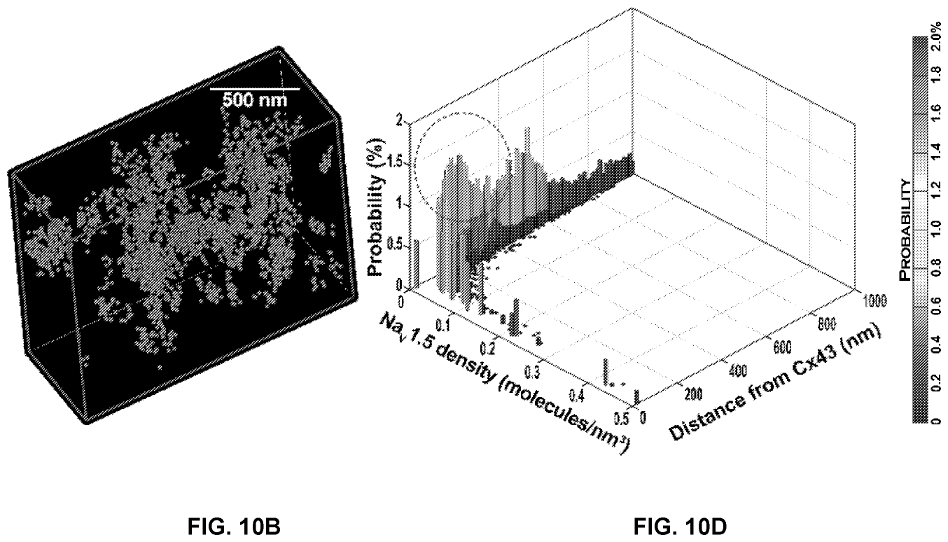

Despite its high resolution, STED microscopy still has limited ability to assess protein density. In any fluorescence image, intensity is determined by a combination of the density of fluorescently-labeled proteins and the number of photons emitted by each. In order to obtain orthogonal validation of the STED results and overcome this limitation, STORM single molecule localization microscopy and STORM-RLA machine learning-based cluster analysis were used. By localizing individual molecules, STORM offers the unique ability to assess relative differences in protein density between different ID regions. FIG. 8 shows representative three-dimensional en face views of atrial IDs obtained by STORM from untreated control hearts: Naᵥ1.5 can be observed as clusters, occurring in close proximity to Cx43 and within N-cad-rich regions, whereas β1 was localized near Cx43 clusters and throughout N-cad-free ID regions. In VEGF-treated hearts, Naᵥ1.5 and β1 clusters appeared more diffuse and were shifted away from Cx43 and N-cad clusters (FIG. 9). Close-up views of Cx43 clusters and associated Naᵥ1.5 clusters supported these findings (FIG. 10A, 10B). STORM data were quantitatively analyzed using STORM-RLA to determine the percent of total Naᵥ1.5/β1 signal at the ID, which was localized within Cx43-adjacent perinexal sites (100 nm from Cx43 clusters) and at N-cad-rich plicate ID sites (FIG. 10E). Additionally, signal enrichment ratio, defined as the ratio of Naᵥ1.5/β1 molecular density at these sites vs. the density at other ID sites was also calculated. In control hearts, 59±2% of Naᵥ1.5 was localized within Cx43-adjacent perinexal sites (enrichment ratio: 10.5±0.3) and 35±2% within N-cad-rich plicate ID sites (enrichment ratio: 6.5±0.4). In contrast, β1 displayed a marked preference for Cx43-adjacent perinexal sites (69±4% of ID-localized β1, enrichment ratio: 10.7±1.9) in comparison to N-cad-rich plicate ID sites (14±3% of ID-localized β1). In VEGF treated hearts, Naᵥ1.5 density was significantly reduced at both Cx43-adjacent perinexal sites (32±3% of signal, enrichment ratio: 6.9±0.8) and N-cad-rich plicate ID sites (26±3% of signal, enrichment ratio: 4.6±0.4). Likewise, β1 density was also reduced at Cx43-adjacent perinexal sites (49±3% of signal, enrichment ratio: 5.4±0.7) without significant changes at N-cad-rich plicate ID sites. Overall, the STORM-RLA results indicated dynamic reorganization of ID-localized Naᵥ1.5 and β1 following VEGF treatment.

Discussion

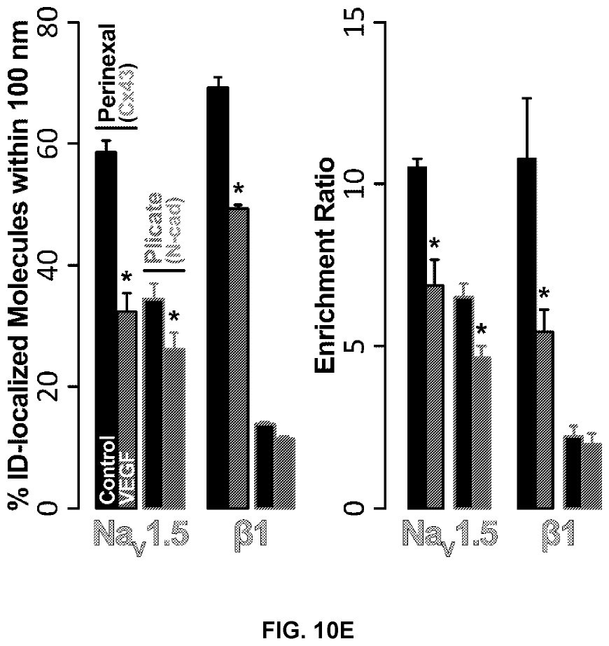

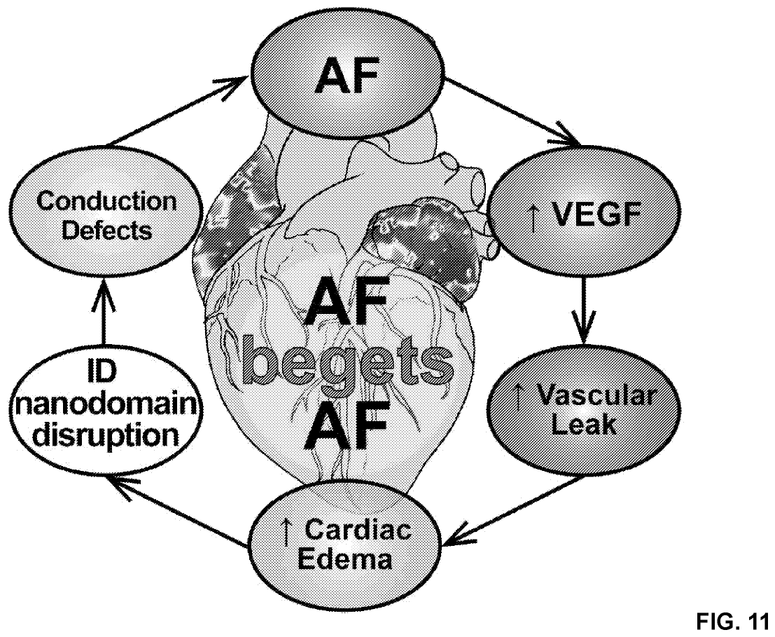

Patients with new-onset AF show elevated levels of VEGF (Li J, et al. Heart rhythm. 2010 7:438-44; Ogi H, et al. Circulation journal. 2010 74:1815-21; Scridon A, et al. Europace. 2012 14:948-53; Seko Y, et al. Jpn Heart J. 2000 41:27-32; Smorodinova N, et al. PloS one. 2015 10:e0129124), a cytokine that promotes vascular leak. Indeed, inflammation, vascular leak, and associated tissue edema are common sequelae of AF (Weis S M. Curr Opin Hematol. 2008 15:243-9; Li J, et al. Heart rhythm. 2010 7:438-44; Ogi H, et al. Circulation journal. 2010 74:1815-21; Scridon A, et al. Europace. 2012 14:948-53; Seko Y, et al. Jpn Heart J. 2000 41:27-32; Gramley F, et al. Cardiovasc Pathol. 2010 19:102-11; Chung N A, et al. Stroke. 2002 33:2187-91), and are emerging as proarrhythmic factors. In previous studies in the ventricles, myocardial edema acutely (within minutes) disrupted ID nanodomains, slowed conduction, and precipitated arrhythmias (Veeraraghavan R, et al. Pflugers Arch. 2015 467:2093-2105; Veeraraghavan R, et al. Pflugers Arch. 2016 468:1651-61; Veeraraghavan R, et al. Am J Physiol Heart Circ Physiol. 2012 302(1):H278-86). Interestingly, patients with AF also evidence swelling of ID nanodomains (Raisch T B, et al. Front Physiol. 2018) and conduction slowing has been linked to AF in human patients (Zheng Y, et al. Clin Physiol Funct Imaging. 2017 37:596-601; Lalani G G, et al. J Am Coll Cardiol. 2012 59:595-606). However, the mechanism by which tissue edema due to vascular leak precipitates AF is unknown. Therefore, the hypothesis that VEGF may acutely promote atrial arrhythmias was tested by disrupting ID nanodomains and compromising atrial conduction (FIG. 11). As disclosed herein, VEGF insult acutely induces ID nanodomain swelling and translocation of sodium channel subunits from these sites, thereby, generating a substrate for slowed atrial conduction, and atrial arrhythmias.

Cytokines such as VEGF, which induce vascular leak, have been shown to have a multitude of other impacts, including directly reducing the expression of Cx43 in cardiac myocytes (Dhein S, et al. Biol Cell. 2002 94:409-22; Pimentel R C, et al. Circulation research. 2002 90:671-7; Fernandez-Cobo M, et al. Cytokine. 1999 11:216-24; Herve J C and Dhein S. Adv Cardiol. 2006 42:107-31; Salameh A, et al. Eur J Pharmacol. 2004 503:9-16; Sawaya S E, et al. Am J Physiol Heart Circ Physiol. 2007 292:H1561-7). In contrast, Western blots indicated no change in the expression of Cx43 or Na⁺ channel subunits, and a slight increase in Cx40 expression following acute VEGF insult. The apparent divergence of our results from the aforementioned studies may reflect the much longer time courses (>4 hours) involved in those compared to this study (<1 hour). Overall, the data suggest that reduced expression of ID proteins cannot explain the rapid proarrhythmic impact of VEGF in these experiments.

In previous studies, acute interstitial edema induced swelling of the perinexus, a GJ-adjacent ID nanodomain, and brought about conduction slowing and spontaneous arrhythmias within 10 minutes (Veeraraghavan R, et al. Pflugers Arch. 2015 467:2093-2105; Veeraraghavan R, et al. Pflugers Arch. 2016 468:1651-61; Veeraraghavan R, et al. Am J Physiol Heart Circ Physiol. 2012 302(1):H278-86). Likewise, elevated extracellular volume, ID nanodomain swelling, and conduction slowing during acute inflammatory response (90 min of exposure to pathophysiological levels of TNFα) (George S A, et al. Front Physiol. 2017 8:334). Consistent with these, the disclosed TEM studies identified significant swelling of ID nanodomains (near both GJs and MJs) following VEGF treatment. Taken together, these results suggest that ID nanodomain swelling may contribute to atrial arrhythmias following acute VEGF insult. Notably, the ultrastructural impact of VEGF in our experiments closely corresponds with observations from human AF patients (Raisch T B, et al. Front Physiol. 2018).

A concomitant impact during acute swelling of ID nanodomains is the translocation of sodium channels from these sites (Veeraraghavan R, et al. Elife. 2018 7). Perinexal swelling was found to decrease local I_{Na} density near GJs, albeit without any change in whole-cell I_{Na} and was sufficient to induce proarrhythmic conduction slowing. These results suggest that the precise localization of sodium channels within the ID may be an important determinant of cardiac electrical propagation. Therefore, super-resolution microscopy was used to test whether VEGF-induced ID remodeling included any reorganization of sodium channel proteins. Overall, STED and STORM both identified Naᵥ1.5 enrichment near Cx43 clusters as well as at N-cad-rich sites, consistent with previous reports (Veeraraghavan R, et al. Pflugers Arch. 2015 467:2093-2105; Veeraraghavan R, et al. Pflugers Arch. 2016 468:1651-610; Veeraraghavan R, et al. Elife. 2018 7; Veeraraghavan R and Gourdie R. Molecular biology of the cell. 2016 27:3583-3590; Leo-Macias A, et al. Nat Commun. 2016 7:10342). In contrast, β1 was preferentially localized near Cx43 and predominantly within N-cad-free ID sites, again in keeping with previous data (Veeraraghavan R, et al. Elife. 2018 7). These data suggest that Naᵥ1.5 at N-cad-rich sites may associate with a different β subunit, an idea which merits future investigation. Importantly, both STED and STORM images revealed changes consistent with decreased Naᵥ1.5 near GJs and MJs in VEGF-treated hearts relative to controls. Quantitative analysis of STED and STORM data revealed a substantial depletion of Naᵥ1.5 from GJ-adjacent perinexal sites, and to a somewhat lesser degree, also from MJ-adjacent sites. Likewise, VEGF treatment also decreased β1 density at GJ-adjacent sites. Overall, these data, along with previously published results (Veeraraghavan R, et al. Elife. 2018 7), suggest that local I_{Na} density at GJ- and MJ-adjacent sites might be decreased following acute VEGF insult. Taken in the context of our TEM results, these data suggest that intermembrane adhesion within ID nanodomains may play a role in retaining sodium channels at these sites. Inhibition of adhesive interactions may enhance lateral diffusion of ion channels within the membrane, resulting in their dispersal from dense clusters. Therefore, disclosed herein is the first direct demonstration of this dynamic remodeling phenomenon.

Taken together, light and electron microscopy results identify two forms of dynamic ID remodeling following acute exposure to VEGF: (1) swelling of the extracellular cleft near GJs and MJs, and (2) translocation of Naᵥ1.5, wherein dense Naᵥ1.5 clusters located near GJs and MJs are redistributed more diffusely. These changes could impair atrial conduction via two, non-mutually exclusive mechanisms: (1) Direct effects on membrane excitability via cooperative activation. The earliest activating Naᵥ1.5 channels promote positive feedback activation of further Naᵥ1.5 channels, when these channels are tightly clustered, and face a restricted extracellular cleft (Hichri E, et al. J Physiol. 2018 Feb. 15 596(4):563-58; Clatot J, et al. Nat Commun. 2017 8:2077). Naᵥ1.5 translocation away from dense clusters into a more diffuse pattern would weaken this effect, and could thereby compromise excitability. (2) Indirect effects on intercellular coupling via ephaptic coupling: When dense Naᵥ1.5 clusters from adjacent cells face each other across a narrow (<30 nm) extracellular cleft, channel activation on one side prompts transient depletion of sodium (positive charge) from the cleft, and subsequent depolarization of the apposed cell's membrane, activating its Naᵥ1.5 channels (Veeraraghavan R, et al. Am J Physiol Heart Circ Physiol. 2014 Mar. 1 306(5):H619-27; Veeraraghavan R, et al. FEBS Lett. 2014 Apr. 17 588(8):1244-8; Veeraraghavan R, et al. Cell Commun Adhes. 2014 Jun. 21(3):161-7; Veeraraghavan R and Radwanski P B. J Physiol. 2018 596:549-550). Both nanodomain swelling and the more diffuse reorganization of Naᵥ1.5 would weaken local electrochemical transients within ID nanodomains, and could thereby impair atrial conduction (Veeraraghavan R, et al. Pflugers Arch. 2015 467:2093-2105; Veeraraghavan R, et al. Pflugers Arch. 2016 468:1651-61; Veeraraghavan R, et al. Elife. 2018 7; Mori Y, et al. Proc Natl Acad Sci USA. 2008 Apr. 29 105(17):6463-8; Kucera J P, et al. Circulation research. 2002 91:1176-82; Lin J and Keener J P. Proc Natl Acad Sci USA. 2010 107:20935-40). Notably, based on their structural properties, both perinexi and plicate nanodomains would support cooperative activation but only perinexi are predicted to support ephaptic coupling (Mori Y, et al. Proc Natl Acad Sci USA. 2008 Apr. 29 105(17):6463-8; Lin J and Keener J P. IEEE Trans Biomed Eng. 2013 60:576-82). However, since VEGF impacted both locations simultaneously, these results do not delineate the relative contributions of the two mechanisms, or indeed of the two different ID nanodomains. The totality of structural and functional results indicate that VEGF can acutely induce proarrhythmic conduction slowing, and likely does so by disrupting ID nanodomains (FIG. 11).

The disclosed results, identifying acute remodeling of ID nanodomains as an arrhythmia mechanism, have important implications for our broader understanding of arrhythmia substrates. Classically, structural arrhythmia substrates are viewed as being permanent (e.g. an infarct), while functional substrates are thought to be dynamic (e.g. a line of block resulting from repolarization heterogeneities). However, vascular leak-induced edema and consequent nanodomain remodeling, as demonstrated here, may represent a dynamic and transient structural arrhythmic substrate. This may contribute to the intermittent nature of arrhythmias in pathologies such as AF in the early stages. The results presented here also have important implications for the treatment of AF. First, they suggest that therapies which mitigate cytokine-induced vascular leak may be effective in preventing atrial arrhythmias. Second, they suggest that direct targeting of ID nanodomains to prevent swelling and sodium channel translocation could also be an effective antiarrhythmic strategy.

In summary, VEGF, at levels occurring in AF patients, can acutely promote atrial arrhythmias and sodium channel clusters at the ID can undergo dynamic reorganization. Importantly, disclosed herein is a new mechanism for atrial arrhythmias, wherein dynamic disruption of ID nanodomains, secondary to VEGF-induced vascular leak, induces proarrhythmic slowing of atrial conduction. This mechanism may contribute to the genesis and progression of AF in the early stages and help explain the link between inflammation and AF. Vascular leak and ID nanodomains are therefore potential therapeutic targets for the treatment and prevention of AF in the early stages.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating a cardiac arrhythmia caused by inflammation-induced vascular leak in a subject, comprising administering to the subject a therapeutically effective amount of a gap junction or pannexin channel inhibitor in an amount effective to preserve barrier function.

2. The method of claim 1, wherein the cardiac arrhythmia comprises an atrial fibrillation (AF).

3. The method of claim 1, wherein the cardiac arrhythmia comprises a reentrant ventricular arrhythmia.

4. The method of claim 1, wherein the gap junction inhibitor is a connexin43 hemichannel inhibitor.

5. The method of claim 4, wherein the connexin43 hemichannel inhibitor is a polypeptide comprising from 4 to 30 contiguous amino acids of the carboxy-terminus of the alpha Connexin.

6. The method of claim 5, wherein the connexin43 hemichannel inhibitor is a polypeptide comprising the amino acid sequence of any one of SEQ ID NOs:2-15.

7. The method of claim 1, wherein the gap junction inhibitor is a mefloquine.

8. The method of claim 1, wherein the pannexin-1 channel inhibitor comprises a Panx1-IL2 peptide.

9. The method of claim 8, wherein the pannexin-1 channel inhibitor comprises the amino acid sequence SEQ ID NO:39 or SEQ ID NO:40.

10. The method of claim 1, wherein the pannexin-1 channel inhibitor comprises A spironolactone.

11. The method of claim 1, wherein the subject has paroxysmal AF.

12. The method of claim 1, further comprising assaying a sample from the subject for a serum biomarker of arrhythmias caused by inflammation-induced vascular leak, wherein detection of the biomarker is an indication that the subject has a cardiac arrhythmia caused by inflammation-induced vascular leak, wherein the biomarker comprises an ectodomain of the sodium channel auxiliary subunit β1.

13. The method of claim 12, wherein the β1 ectodomain comprises the amino acid sequence KRRSETTAETFTEWTFR (SEQ ID NO:1).

14. The method of claim 13, wherein the serum biomarker is detected using an antibody that selectively binds SEQ ID NO:1.


SEQUENCE LISTING (38 sequences)

SEQ ID NO: 1 - protein, 20 aa, Artificial Sequence
  Synthetic Construct
PSSRASSRASSRPRPDDLEI

SEQ ID NO: 2 - protein, 9 aa, Artificial Sequence
  Synthetic Construct
RPRPDDLEI

SEQ ID NO: 3 - protein, 9 aa, Artificial Sequence
  Synthetic Construct
RPRPDDLEV

SEQ ID NO: 4 - protein, 9 aa, Artificial Sequence
  Synthetic Construct
RPRPDDVPV

SEQ ID NO: 5 - protein, 9 aa, Artificial Sequence
  Synthetic Construct
KARSDDLSV

SEQ ID NO: 6 - protein, 19 aa, Artificial Sequence
  Synthetic Construct
SSRASSRASSRPRPDDLEV

SEQ ID NO: 7 - protein, 9 aa, Artificial Sequence
  Synthetic Construct
RPKPDDLEI

SEQ ID NO: 8 - protein, 19 aa, Artificial Sequence
  Synthetic Construct
SSRASSRASSRPKPDDLEI

SEQ ID NO: 9 - protein, 9 aa, Artificial Sequence
  Synthetic Construct
RPKPDDLDI

SEQ ID NO: 10 - protein, 19 aa, Artificial Sequence
  Synthetic Construct
SSRASSRASSRPRPDDLDI

SEQ ID NO: 11 - protein, 19 aa, Artificial Sequence
  Synthetic Construct
SSRASTRASSRPRPDDLEI

SEQ ID NO: 12 - protein, 9 aa, Artificial Sequence
  Synthetic Construct
RPRPEDLEI

SEQ ID NO: 13 - protein, 19 aa, Artificial Sequence
  Synthetic Construct
SSRASSRASSRPRPEDLEI

SEQ ID NO: 14 - protein, 9 aa, Artificial Sequence
  Synthetic Construct
GDGKNSVWV

SEQ ID NO: 15 - protein, 18 aa, Artificial Sequence
  Synthetic Construct
GQKPPSRPSSSASKKLYV

SEQ ID NO: 16 - protein, 120 aa, Artificial Sequence
  Synthetic Construct
KTDPYSHSGTMSPSKDCGSPKYAYYNGCSSPTAPLSPMSPPGYKLVTGDRNNSSCRNYNKQASEQNWANYSAEQNRMGQAGSTISNSHAQPFDFADEHQNTKKLASGHELQPLTIVDQRP

SEQ ID NO: 17 - protein, 36 aa, Artificial Sequence
  Synthetic Construct
RQPKIWFPNRRKPWKKPSSRASSRASSRPRPDDLEI

SEQ ID NO: 18 - protein, 25 aa, Artificial Sequence
  Synthetic Construct
RQPKIWFPNRRKPWKKRPRPDDLEI

SEQ ID NO: 19 - protein, 25 aa, Artificial Sequence
  Synthetic Construct
RQPKIWFPNRRKPWKKRPRPDDLEV

SEQ ID NO: 20 - protein, 25 aa, Artificial Sequence
  Synthetic Construct
RQPKIWFPNRRKPWKKRPRPDDVPV

SEQ ID NO: 21 - protein, 25 aa, Artificial Sequence
  Synthetic Construct
RQPKIWFPNRRKPWKKKARSDDLSV

SEQ ID NO: 22 - protein, 20 aa, Artificial Sequence
  Synthetic Construct
GRKKRRQRPPQRPRPDDLEI

SEQ ID NO: 23 - protein, 25 aa, Artificial Sequence
  Synthetic Construct
RQIKIWFQNRRMKWKKRPRPDDLEI

SEQ ID NO: 24 - protein, 25 aa, Artificial Sequence
  Synthetic Construct
RQIAIWFQNRRMKWAARPRPDDLEI

SEQ ID NO: 25 - protein, 18 aa, Artificial Sequence
  Synthetic Construct
RKKRRQRRRRPRPDDLEI

SEQ ID NO: 26 - protein, 30 aa, Artificial Sequence
  Synthetic Construct
TRSSRAGLQFPVGRVHRLLRKRPRPDDLEI

SEQ ID NO: 27 - protein, 35 aa, Artificial Sequence
  Synthetic Construct
GWTLNSAGYLLGKINKALAALAKKILRPRPDDLEI

SEQ ID NO: 28 - protein, 27 aa, Artificial Sequence
  Synthetic Construct
KLALKLALKALKAALKLARPRPDDLEI

SEQ ID NO: 29 - protein, 25 aa, Artificial Sequence
  Synthetic Construct
AAVALLPAVLLALLAPRPRPDDLEI

SEQ ID NO: 30 - protein, 19 aa, Artificial Sequence
  Synthetic Construct
VPMLKPMLKERPRPDDLEI

SEQ ID NO: 31 - protein, 36 aa, Artificial Sequence
  Synthetic Construct
ANLGYWLLALFVTMWTDVGLCKKRPKPRPRPDDLEI

SEQ ID NO: 32 - protein, 27 aa, Artificial Sequence
  Synthetic Construct
LLIILRRRIRKQAHAHSKRPRPDDLEI

SEQ ID NO: 33 - protein, 30 aa, Artificial Sequence
  Synthetic Construct
KETWWETWWTEWSQPKKKRKVRPRPDDLEI

SEQ ID NO: 34 - protein, 27 aa, Artificial Sequence
  Synthetic Construct
RGGRLSYSRRRFSTSTGRRPRPDDLEI

SEQ ID NO: 35 - protein, 24 aa, Artificial Sequence
  Synthetic Construct
SDLWEMMMVSLACQYRPRPDDLEI

SEQ ID NO: 36 - protein, 21 aa, Artificial Sequence
  Synthetic Construct
TSPLNIHNGQKLRPRPDDLEI

SEQ ID NO: 37 - protein, 10 aa, Artificial Sequence
  Synthetic Construct
KYPIVEQYLK

SEQ ID NO: 38 - protein, 19 aa, Artificial Sequence
  Synthetic Construct
KYPIVEQYLKYGRKKQRRR